SEQUENCE LISTING (2 sequences)

SEQ ID NO: 1 - DNA, 31 bp, Artificial Sequence
  DNA, artificial, single stranded
cacgtccagatcaatgactatgagcagtaca

SEQ ID NO: 2 - DNA, 32 bp, Artificial Sequence
  DNA, artificial, single stranded
attatttatttatthmcgtattatttattatt

(19) United States
(12) Patent Application Publication
(10) Pub. No.: US 2025/0283145 A1
(43) Pub. Date: Sep. 11, 2025

(54) METHODS FOR BASE-LEVEL DETECTION OF METHYLATION IN NUCLEIC ACIDS

(71) Applicants: Roche Sequencing Solutions, Inc., Pleasanton, CA (US); KAPA BIOSYSTEMS, INC., Wilmington, DE (US); ROCHE DIAGNOSTICS GMBH, Mannheim (DE)

(72) Inventors: Frank Bergmann, Iffeldorf (DE); Shwu shin Chang, Dublin, CA (US); Peter Crisalli, Pleasanton, CA (US); Abre De Beer, Western Cape (ZA); Dieter Heindl, Muenchen (DE); Omid Khakshoor, Lathrop, CA (US); David Lawrence Penkler, San Ramon, CA (US); Jo-Anne Elizabeth Penkler, San Ramon, CA (US); Martin Ranik, Dublin, CA (US); Meng Taing, Hayward, CA (US)

(21) Appl. No.: 18/264,302

(22) PCT Filed: Feb. 8, 2022
(86) PCT No.: PCT/EP2022/052979

§ 371 Date: Aug. 4, 2023

Related U.S. Application Data
(60) Provisional application No. 63/147,301, filed on Feb. 9, 2021.
(60) Provisional application No. 63/150,019, filed on Feb. 16, 2021.
(60) Provisional application No. 63/191,079, filed on May 20, 2021.

(51) Int. Cl. C12Q 1/6806; C12Q 1/26; C12Q 1/48; C12Q 1/6869

(52) U.S. Cl. CPC C12Q 1/6806; C12Q 1/26; C12Q 1/485; C12Q 1/6869

(57) ABSTRACT

The invention includes improved methods and compositions for detecting methylation in nucleic acids. In particular, the disclosure is directed to methods of converting 5-hydroxymethylcytosine (5hmC) and/or 5-methylcytosine (5mC) to Thymine (T). In addition, the disclosure is also directed to methods of detecting 5hmC and/or 5mC in a sample.

### FIELD OF THE INVENTION

The invention related to the field of nucleic acid-based diagnostics. More specifically, the invention related to a method of detecting epigenetic modification in nucleic acids, wherein the epigenetic modifications may have biological and clinical significance.

### SEQUENCE LISTING

The Instant patent application contains a Sequence Listing, which has been submitted electronically in XML format and is hereby incorporated by reference in its entirety. The XML copy, created Aug. 2, 2024, is named “20240802_P36560-US-1_Revised_Sequence_Listing,” and is 3,664 bytes in size.

### BACKGROUND OF THE INVENTION

Epigenetic modifications and in particular, DNA methylation play a role in development and in pathological processes. Detecting methylation comprises detecting a modified cytosine base (methyl and hydroxymethyl cytosine (5mC and 5hmC)) in nucleic acids. Until recently, the gold standard of detecting methylation involved treating DNA with bisulfite. The treatment would convert unmethylated cytosines (C) to uracils (U) while methylated cytosines (5mC and 5hmC) would remain intact. The change of C to U could then be detected e.g., by nucleic acid sequencing. Unfortunately, bisulfite treatment leads to degradation of large portion of sample DNA. Alternative, less harsh methods for the detection of methylated cytosines include enzymatic treatment with ten-eleven translocation (TET) dioxygenases and detecting any one of the oxidation products. One particular method called TAPS (TET-assisted pyridine-borane sequencing) involves oxidation of methylated cytosines in nucleic acid with TET and co-catalysts (e.g., Fe(II) ions and alpha-ketoglutarate) and treatment of oxidation products with borane derivatives to form dihydrouracil (DHU) which is read as T during sequencing, see Liu, Y., et al. (2019) Bisulfite-free direct detection of 5-methylcytosine and 5-hydroxymethylcytosine at base resolution. Nat. Biotechnol. 37, 424-429. Other methods also utilize TET but offer alternatives to borane reduction.

In one example, oxidation products can be reacted with malononitrile to form an adduct also read as T during sequencing, see Zhu C., et al., (2017) Single-Cell 5-Formylcytosine Landscapes of Mammalian Early Embryos and ESCs at Single-Base Resolution, Cell Stem Cell, 20:720-731.e5. Malononitrile reacts exclusively with 5-formylcytosine (5fC). Another method of detecting 5fC is with a Wittig reagent in an organic solvent, and then irradiating with ultraviolet light. The products of the reaction are detected using fluorescence recognition technology as described in International Patent Publication No. WO2020155742.

Unfortunately, the newer methods face obstacles before they can be widely adopted by clinical laboratories: the chemical reactions in TAPS and malononitrile method either require high temperatures (70° C.) or take multiple days to complete. There is a need for a rapid and convenient methylation detection assay that could be deployed in clinical labs.

Unfortunately, the currently used oxidation methods (such as TET in the presence of Fe(II) ions and alpha-ketoglutarate) yield a mixture of 5-carboxycytosine (5caC) and 5-formylcytosine (5fC). To enable accurate base-level detection of cytosine methylation, there is a need for an enzymatic method of selectively oxidizing methylated cytosines and forming preferentially or exclusively 5fC for downstream detection procedures.

### SUMMARY OF THE INVENTION

In some embodiments, the invention is a method of detecting a 5-formyl cytosine (5fC) nucleotide in a nucleic acid, the method comprising: (i) forming a reaction mixture by contacting a sample containing a nucleic acid comprising 5fC with a composition comprising a compound of formula R₁—CH₂—CN capable of reacting with 5fC in the nucleic acid to form an adduct according to the reaction scheme

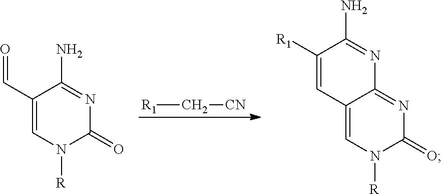

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl, or heteroaryl; (ii) incubating the reaction mixture for less than 3 hours wherein at least 90% of 5fC has formed the adduct; (iii) sequencing the nucleic acid from the reaction mixture to obtain a test sequence wherein the adduct is read as thymine (T) during sequencing; and (iii) comparing the test sequence with a reference sequence, wherein a transition from a cytosine (C) in the reference sequence to a thymine (T) in the corresponding position in the test sequence indicates the presence of 5fC in the nucleic acid. In some embodiments, R1 is a cyano group (CN). In some embodiments, the composition comprising the compound of formula R₁—CH₂—CN contains an organic acid moiety. In some embodiments, the organic acid has a formula R—COOH and R is selected from C1-C30 linear or branched alkyl, C1-C30 linear or branched alkenyl, and C1-C30 linear or branched alkynyl. In some embodiments, the organic acid is acetic acid. In some embodiments, the composition comprising the compound of formula R₁—CH₂—CN is present in a non-aqueous solvent. In some embodiments, the non-aqueous solvent has a formula R—OH wherein R is selected from C1-C30 linear or branched alkyl, C1-C30 linear or branched alkenyl, C1-C30 linear or branched alkynyl, cycloalkyl, aryl, or heteroaryl. In some embodiments, the non-aqueous solvent is methanol or ethanol at 10-100%, e.g., 90-100%. In some embodiments, the reaction mixture further comprises a compound of formula RₓNH_{y} wherein x and y are 0, 1, 2 or 3 so that x+y=3, and each R is independently selected from C1-C30 linear or branched alkyl, C1-C30 linear or branched alkenyl, C1-C30 linear or branched alkynyl, cycloalkyl, aryl, or heteroaryl. In some embodiments, RₓNH_{y} is triethanolamine. In some embodiments, the reaction mixture is incubated for 1 hour. In some embodiments, prior to sequencing in step (iii), the nucleic acid is amplified, e.g., with a B-family polymerase efficiently incorporating an adenine (A) nucleotide opposite the adduct. In some embodiments, sequencing in step (iii) is by sequencing-by-synthesis (SBS) method, e.g., with a nanopore.

In some embodiments, the nucleic acid comprising 5fC is obtained by contacting the nucleic acid comprising methylated cytosine with a composition comprising a ten-eleven-translocation (TET) dioxygenase and 5-100 μM of a Fe(II) ion at pH 7-8. In some embodiments, the composition comprises a ten-eleven-translocation (TET) dioxygenase and 5-10 μM of a Fe(II) ion at pH 8. In some embodiments, the composition comprises a ten-eleven-translocation (TET) dioxygenase and 80-100 UM of a Fe(II) ion at pH 7. In some embodiments, the Fe(II) ion is produced by contacting the sample with a compound selected from FeSO₄, (NH₄)₂Fe(SO₄)₂, FeSO₄7H₂O, (NH₄)₂Fe(SO₄)₂6H₂O and FeCl₂. In some embodiments, the composition further comprises one or more of ascorbic acid, alpha-ketoglutarate and a reducing agent. In some embodiments, the nucleic acid comprising 5fC is obtained by contacting the nucleic acid comprising methylated cytosine with a composition comprising Cu(II) compound and 2,2,6,6-tetramethylpiperidine-1-oxyl (TEMPO). In some embodiments, the nucleic acid comprising 5fC is obtained by contacting the nucleic acid comprising methylated cytosine with a potassium ruthenium salt selected from potassium ruthenate (K₂RuO₄) and potassium perruthenate (KRuO₄).

In some embodiments, the invention is a method of detecting a methylated cytosine (C) nucleotide in a nucleic acid, the method comprising: (i) forming a reaction mixture by contacting a sample containing a nucleic acid comprising 5-methyl cytosine (5mC) and/or 5-hydroxymethyl cytosine (5hmC) with a composition comprising a ten-eleven-translocation (TET) dioxygenase capable of converting 5mC and 5hmC in the nucleic acid into 5-formyl cytosine (5fC) and a compound of formula R₁—CH₂—CN capable of reacting with 5fC in the nucleic acid to form an adduct according to the reaction scheme

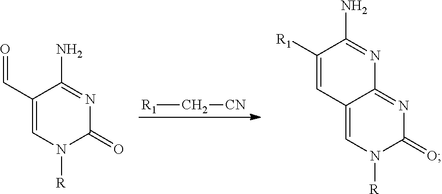

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl, or heteroaryl; (ii) incubating the reaction mixture for less than 3 hours wherein at least 90% of 5fC has formed the adduct; (iii) sequencing the nucleic acid from the reaction mixture to obtain a test sequence wherein the adduct is read as thymine (T) during sequencing; and (iii) comparing the test sequence with a reference sequence, wherein a transition from a cytosine (C) in the reference sequence to a thymine (T) in the corresponding position in the test sequence indicates the presence of a methylated cytosine in the nucleic acid. In some embodiments, R1 is a cyano group (CN) and the composition added in step (i) contains a non-aqueous solvent having a formula R—OH wherein R is selected from C1-C30 linear or branched alkyl, C1-C30 linear or branched alkenyl, C1-C30 linear or branched alkynyl, cycloalkyl, aryl, or heteroaryl and the concentration of the solvent in the reaction mixture of at least 90%. In some embodiments, R1 is a cyano group (CN) and the composition added in step (i) contains ethanol or methanol at the concentration of at least 90% in the reaction mixture, and further comprises tri-ethanolamine.

In some embodiments, the invention is a method of detecting a methylated cytosine nucleotide in a nucleic acid, the method comprising: (i) ligating adaptors to a nucleic acid comprising 5-methyl cytosine (5mC) and/or 5-hydroxymethyl cytosine (5hmC), wherein adaptors comprise amplification primer binding sites; (ii) forming a reaction mixture by contacting the sample containing adaptor-ligated nucleic acid with a ten-eleven-translocation (TET) dioxygenase capable of converting 5mC and 5hmC in the nucleic acid into 5-formyl cytosine (5fC);
(iii) contacting the reaction mixture with a compound of formula R₁—CH₂—CN capable of reacting with 5fC in the nucleic acid to form an adduct according to the reaction scheme

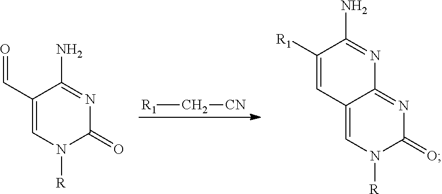

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl, or heteroaryl; (iv) incubating the reaction mixture for less than 3 hours wherein at least 90% of 5fC has formed the adduct; (v) amplifying the adapted nucleic acids utilizing a DNA polymerase and primers capable of binding to the primer-binding sites, wherein the DNA polymerase reads the adduct as thymine (T) during amplification; (vi) sequencing the amplified nucleic acid to obtain a test sequence; (vii) comparing the test sequence with a reference sequence, wherein a transition from a cytosine (C) in the reference sequence to a thymine (T) in the corresponding position in the test sequence indicates the presence of a methylated cytosine in the nucleic acid. In some embodiments, R1 is a cyano group (CN) and the composition added in step (i) contains a non-aqueous solvent having a formula R—OH wherein R is selected from C1-C30 linear or branched alkyl, C1-C30 linear or branched alkenyl, C1-C30 linear or branched alkynyl, cycloalkyl, aryl, or heteroaryl, and the concentration of the solvent in the reaction mixture of at least 90%. In some embodiments, R1 is a cyano group (CN) and the composition added in step (i) contains ethanol or methanol at the concentration of at least 90% in the reaction mixture, and further comprises tri-ethanolamine.

In some embodiments, the invention is a kit for detecting 5-formyl cytosine (5fC) in a nucleic acid in under 3 hours, the kit comprising an ethanol solution of malononitrile. In some embodiments, the kit further comprises one or more of the following: nucleic acid sequencing reagents, nucleic acid amplification reagents, nucleic acid purification reagents, a solution of acetic acid, a solution of triethanolamine and instructions on reacting 5fC in nucleic acid with malononitrile in the presence of organic acids and alkylamines.

In some embodiments, the invention is a kit for detecting methylated cytosine nucleotides in a nucleic acid in under 3 hours, the kit comprising a ten-eleven-translocation (TET) dioxygenase enzyme, an ethanol solution of malononitrile and further comprising reagents for nucleic acid purification, amplification and sequencing.

In some embodiments, the invention is a method of detecting a 5-formyl cytosine (5fC) and 5-carboxy cytosine (5caC) nucleotide in a nucleic acid, the method comprising: (i) forming a reaction mixture by contacting a sample containing a nucleic acid comprising 5fC and/or 5caC with a composition comprising a borane derivative; (ii) incubating the reaction mixture for less than 3 hours wherein at least 90% of 5fC and 5caC has been reduced to dihydrouracil (DHU);
(iii) sequencing the nucleic acid from the reaction mixture to obtain a test sequence wherein DHU is read as thymine (T) during sequencing; and (iii) comparing the test sequence with a reference sequence, wherein a transition from a cytosine (C) in the reference sequence to a thymine (T) in the corresponding position in the test sequence indicates the presence of 5fC or 5caC in the nucleic acid. In some embodiments, the borane derivative is picoline borane. In some embodiments, the reaction mixture contains an organic acid moiety. In some embodiments, the organic acid has a formula R—COOH and R is selected from C1-C30 linear or branched alkyl, C1-C30 linear or branched alkenyl, and C1-C30 linear or branched alkynyl. In some embodiments, the organic acid is acetic acid. In some embodiments, the borane derivative is present in a non-aqueous solvent. In some embodiments, the non-aqueous solvent has a formula R—OH wherein R is selected from C1-C30 linear or branched alkyl, C1-C30 linear or branched alkenyl, C1-C30 linear or branched alkynyl, cycloalkyl, aryl, or heteroaryl. In some embodiments, the non-aqueous solvent is methanol or ethanol. In some embodiments, the reaction mixture is incubated for 1 hour. In some embodiments, prior to sequencing in step (iii), the nucleic acid is amplified, e.g., with a B-family polymerase efficiently incorporating an adenine (A) nucleotide opposite DHU. In some embodiments, sequencing in step (iii) is by sequencing-by-synthesis (SBS) method, e.g., with a nanopore. In some embodiments, the nucleic acid comprising 5fC and/or 5caC is obtained by contacting the nucleic acid comprising methylated cytosine with a composition comprising a ten-eleven-translocation (TET) dioxygenase and 5-100 μM of a Fe(II) ion at pH 7-8. In some embodiments, the composition comprises a ten-eleven-translocation (TET) dioxygenase and 5-10 UM of a Fe(II) ion at pH 8. In some embodiments, the composition comprises a ten-eleven-translocation (TET) dioxygenase and 80-100 μM of a Fe(II) ion at pH 7. In some embodiments, the Fe(II) ion is produced by contacting the sample with a compound selected from FeSO₄, (NH₄)₂Fe(SO₄)₂, FeSO₄7H₂O, (NH₄)₂Fe(SO₄)₂6H₂O and FeCl₂. In some embodiments, the composition further comprises one or more of ascorbic acid, alpha-ketoglutarate and a reducing agent. In some embodiments, the nucleic acid comprising 5fC is obtained by contacting the nucleic acid comprising methylated cytosine with a composition comprising Cu(II) compound and 2,2,6,6-tetramethylpiperidine-1-oxyl (TEMPO). In some embodiments, the nucleic acid comprising 5fC is obtained by contacting the nucleic acid comprising methylated cytosine with a potassium ruthenium salt selected from potassium ruthenate (K₂RuO₄) and potassium perruthenate (KRuO₄).

In some embodiments, the invention is a method of detecting a methylated cytosine (C) nucleotide in a nucleic acid, the method comprising: (i) forming a reaction mixture by contacting a sample containing a nucleic acid comprising 5-methyl cytosine (5mC) and/or 5-hydroxymethyl cytosine (5hmC) with a composition comprising a ten-eleven-translocation (TET) dioxygenase capable of converting 5mC and 5hmC in the nucleic acid into 5-formyl cytosine (5fC) and 5-carboxy cytosine (5caC) and a borane derivative in a non-aqueous solvent;

(ii) incubating the reaction mixture for less than 3 hours wherein at least 90% of 5fC and 5caC has been reduced to dihydrouracil (DHU); (iii) sequencing the nucleic acid from the reaction mixture to obtain a test sequence wherein the DHU is read as thymine (T) during sequencing; and (iv) comparing the test sequence with a reference sequence, wherein a transition from a cytosine (C) in the reference sequence to a thymine (T) in the corresponding position in the test sequence indicates the presence of a methylated cytosine in the nucleic acid. In some embodiments, the borane derivative is selected from pyridine borane, 2-picoline borane (pic-BH3), borane, sodium borohydride, sodium cyanoborohydride, and sodium triacetoxyborohydride and the non-aqueous solvent is selected from methanol and methanol and the reaction mixture further comprises acetic acid.

In some embodiments, the invention is a method of detecting a methylated cytosine nucleotide in a nucleic acid, the method comprising: (i) ligating adaptors to a nucleic acid comprising 5-methyl cytosine (5mC) and/or 5-hydroxymethyl cytosine (5hmC), wherein adaptors comprise amplification primer binding sites; (ii) forming a reaction mixture by contacting the sample containing adaptor-ligated nucleic acid with a ten-eleven-translocation (TET) dioxygenase capable of converting 5mC and 5hmC in the nucleic acid into 5-formyl cytosine (5fC) and 5-carboxycytosine (5caC); (iii) contacting the reaction mixture with a borane derivative in a non-aqueous solvent; (iv) incubating the reaction mixture for less than 3 hours wherein at least 90% of 5fC and 5caC has been reduced to dihydrouracil (DHU); (v) amplifying the adapted nucleic acids utilizing a DNA polymerase and primers capable of binding to the primer-binding sites, wherein the DNA polymerase reads DHU as thymine (T) during amplification; (vi) sequencing the amplified nucleic acid to obtain a test sequence; (vii) comparing the test sequence with a reference sequence, wherein a transition from a cytosine (C) in the reference sequence to a thymine (T) in the corresponding position in the test sequence indicates the presence of a methylated cytosine in the nucleic acid. In some embodiments, the borane derivative is selected from pyridine borane, 2-picoline borane (pic-BH3), borane, sodium borohydride, sodium cyanoborohydride, and sodium triacetoxyborohydride and the non-aqueous solvent is selected from methanol and methanol and the reaction mixture further comprises acetic acid.

In some embodiments, the invention is a kit for detecting 5-formyl cytosine (5fC) and 5-carboxy cytosine (5caC) in a nucleic acid in under 3 hours, the kit comprising a borane derivative in an ethanol solution. In some embodiments, the borane derivative is selected from pyridine borane, 2-picoline borane (pic-BH3), borane, sodium borohydride, sodium cyanoborohydride, and sodium triacetoxyborohydride. In some embodiments, the kit further comprises one or more of the following: nucleic acid sequencing reagents, nucleic acid amplification reagents, nucleic acid purification reagents, a solution of acetic acid, and instructions on reacting 5fC and 5caC in nucleic acid with a borane compound in the presence of organic acids.

In some embodiments, the invention is a kit for detecting methylated cytosine nucleotides in a nucleic acid in under 3 hours, the kit comprising a ten-eleven-translocation (TET) dioxygenase enzyme, an ethanol solution of a borane derivative and further comprising reagents for nucleic acid purification, amplification and sequencing.

In some embodiments, the invention is a single-tube method of detecting a 5-formyl cytosine (5fC) nucleotide in a nucleic acid, the method comprising: (i) forming a reaction mixture by contacting a sample containing a nucleic acid comprising 5fC with a composition comprising ten-eleven-translocation (TET) dioxygenase in a solution with a compound of formula R₁—CH₂—CN capable of reacting with 5fC in the nucleic acid to form an adduct according to the reaction scheme

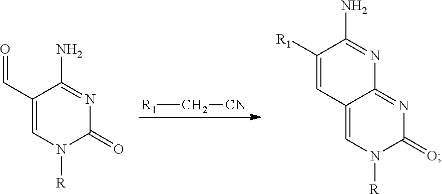

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl, or heteroaryl; (ii) incubating the reaction mixture to form the adduct;
(iii) sequencing the nucleic acid from the reaction mixture to obtain a test sequence wherein the adduct is read as thymine (T) during sequencing; and (iii) comparing the test sequence with a reference sequence, wherein a transition from a cytosine (C) in the reference sequence to a thymine (T) in the corresponding position in the test sequence indicates the presence of 5fC in the nucleic acid. In some embodiments, the compound of formula R₁—CH₂—CN is malononitrile.

In some embodiments, the invention is a method of detecting a tissue of origin of a nucleic acid in a sample, the method comprising detecting the presence and location of methylated cytosines in the nucleic acid by the method disclosed herein, comparing the methylation pattern to the known methylation patterns of several tissues; and identifying the tissue of origin of the nucleic acid in the sample.

In some embodiments, the invention is a method of detecting organ transplant rejection in a transplant recipient, the method comprising obtaining from the transplant recipient a blood sample containing cell-free nucleic acids; detecting the presence and location of methylated cytosines in the cell-free nucleic acid by the method disclosed herein; comparing the methylation pattern to the known methylation patterns of several organs; detecting transplant rejection if the cell-free nucleic acid with the transplanted organ-specific methylation pattern is detected in the sample. In some embodiments, the invention is a method of monitoring for transplant rejection by periodically sampling circulating cell-free DNA and detecting the presence and location of methylated cytosines according to the disclosed herein, measuring changes in the level of cell-free DNA with the transplanted organ-specific methylation pattern, wherein an increase in the level of such cell-free DNA indicates organ transplant rejection.

In some embodiments, the invention is a method of screening for the presence of a cancerous tumor in a patient, the method comprising obtaining from the patient a blood sample containing cell-free nucleic acids; detecting the presence and location of methylated cytosines in the cell-free nucleic acids by the method disclosed herein; comparing the methylation pattern to the known methylation patterns of tumor and non-tumor tissues, and detecting the presence of a tumor if tumor-specific methylation patter is detected. In some embodiments, the invention is a method of monitoring tumor volume in a patient the method comprising periodically sampling circulating cell-free DNA and detecting the presence and location of methylated cytosines by the method disclosed herein, measuring changes in the level of cell-free DNA with the tumor-specific methylation pattern, wherein an increase in the level of such cell-free DNA indicates tumor growth, while a decrease in the level of such cell-free DNA indicates tumor shrinkage. In some embodiments, the invention is a method of monitoring the effectiveness of treatment of cancer in a patient by a method comprising periodically sampling circulating cell-free DNA and detecting the presence and location of methylated cytosines by the method disclosed herein, measuring changes in the level of cell-free DNA with the tumor-specific methylation pattern, wherein an increase in the level of such cell-free DNA indicates that treatment ineffective, while a decrease in the level of such cell-free DNA indicates treatment effectiveness.

In some embodiments, the invention is a method of diagnosis or minimal residual disease (MRD) in a cancer patient the method comprising obtaining from the patient a blood sample comprising cell-free nucleic acids, detecting the presence and location of methylated cytosines in the nucleic acid by the method disclosed herein, comparing the methylation pattern to the known methylation patterns of several tissues; and identifying the tissue of origin of the nucleic acid in the sample.

In some embodiments, the invention is a method of diagnosing an autoimmune disease in a patient the method comprising from the patient a blood sample comprising cell-free nucleic acids, detecting the presence and location of methylated cytosines in the nucleic acid by the method disclosed herein, comparing the methylation pattern to the known methylation patterns of tissues damaged by the immune disease; diagnosing immune disease if such methylation pattern is found.

In some embodiments, the invention is a method detecting the presence and location of methylated cytosines as disclosed herein further comprising prior to contacting the reaction mixture with the TET dioxygenase, the 5-hydroxymethyl cytosine (5hmC) in the nucleic acid is chemically blocked from reacting with TET. In some embodiments, 5hmC is blocked by contacting the reaction mixture with a glucosyltransferase and a glucose moiety. In some embodiments, the reaction mixture is contacted with a beta-glucosyltransferase and UDP glucose.

In some embodiments, the invention is a method of forming 5-formyl cytosine (5fC) in a nucleic acid comprising contacting a reaction mixture containing the nucleic acid including at least one 5-hydroxymethyl cytosine (5hmC) with laccase. In some embodiments, laccase is isolated from a species selected from Hexagonia tenuis, Pleurotis sajor caju, Pleutoris ostreatus, Xylaria polymorpha, Trametes hirsuta, Trametes versicolor and Coprinus spp. In some embodiments, laccase is isolated from a strain selected from Pleurotis sajor caju MTCC-141, Pleutoris ostreatus MTCC-1801, Xylaria polymorpha MTCC-1100, and Trametes hirsuta MTCC-1171. In some embodiments, the reaction mixture further comprises a co-factor, e.g., 2,2,6,6-tetramethylpiperidine-1-oxyl (TEMPO), acetosyringone, syringaldehyde, para-coumaric acid 2,2′-azino-bis(3-ethylbenzothiazoline-6-sulfonate (ABTS), violuric acid (VLA), N-acetyl-N-phenylhydroxylamine (NHA), N-hydroxybenzotriazole (HBT), and N-hydroxyphthalimide (HPI).

In some embodiments, the invention is a method of detecting 5-hydroxymethyl cytosine (5hmC) in nucleic acids comprising the steps of: contacting a sample comprising the nucleic acid comprising 5hmC with laccase under conditions suitable for oxidizing 5hmC into 5-formyl cytosine (5fC); contacting the sample with a composition comprising a compound of formula R₁—CH₂—CN capable of reacting with 5fC in the nucleic acid to form an adduct according to the reaction scheme

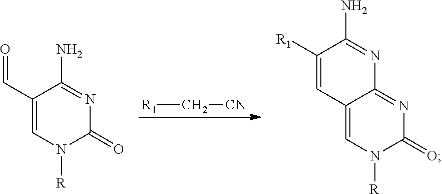

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl under conditions suitable for forming a 5fC adduct; sequencing the nucleic acid from the reaction mixture to obtain a test sequence wherein the adduct is read as thymine (T) during sequencing; and comparing the test sequence with a reference sequence, wherein a transition from a cytosine (C) in the reference sequence to a thymine (T) in the corresponding position in the test sequence indicates the presence of 5hmC in the nucleic acid. In some embodiments, the alkyl substitution may comprise heteroatoms like 0 or N, e.g. —CH₂—CH₂—O—CH₃. In some embodiments, the compound of formula R₁—CH₂—CN is malononitrile. Alternatively, the same conversion can be performed with a Wittig reagent. Then, R1-CH2-CN is replaced by Ph3P=C(R2)CN, wherein R2 is one of hydrogen, cyano, halogen, alkyl and alkyl containing 0, N, halogen, P, S or Si. This is disclosed in WO2020/155742.
   The reaction is as follows:

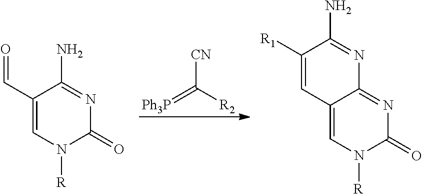

In the following, the above disclosed R1-CH2-CN and the Wittig reagent are defined as “fC Conversion Reagent”, since they are capable of converting a Cytosine into a Thymine equivalent, when serving as a polymerase substrate.
Further, in the following, the reaction product of R1-CH2-CN and the Wittig reagent with 5-formyl-cytosine (5fC) is defined as “5fC adduct” or “adduct” which acts a thymine equivalent when serving as a polymerase substrate.

In some embodiments, the reaction mixture further contains one or more of the following: an organic acid, a non-aqueous solvent and a compound of formula RₓNH_{y} wherein x and y are 0, 1, 2 or 3 so that x+y=3, and each R is independently selected from C1-C6 linear or branched alkyl which may comprise heteroatoms such as O and N, C6-C10-aryl, or 5- or 6-membered heteroaryl. In some embodiments, the organic acid is acetic acid, the non-aqueous solvent is ethanol or methanol and the compound of formula RₓNH_{y} is tri-ethanolamine or piperidine. In other embodiments, the compound is a buffer, e.g. ammonium acetate or Tris. In some embodiments, the non-aqueous solvent is present in the reaction mixture at a concentration of 10-100%, e.g., 90-100%. In some embodiments, the reaction mixture is incubated for 1 hour. In some embodiments, prior to sequencing, the nucleic acid is amplified with a B-family polymerase efficiently incorporating an adenine (A) nucleotide opposite the Thymine equivalent.

In some embodiments, the invention is a method of detecting a methylated cytosine (C) in a nucleic acid, the method comprising: contacting a sample containing a nucleic acid comprising 5-methyl cytosine (5mC) and/or 5-hydroxymethyl cytosine (5hmC) with a ten-eleven-translocation (TET) dioxygenase capable of converting 5mC into 5hmC and with a laccase capable of converting 5hmC into 5-formyl cytosine (5fC); contacting the sample with a compound of formula R₁—CH₂—CN capable of reacting with 5fC in the nucleic acid to form an adduct according to the reaction scheme

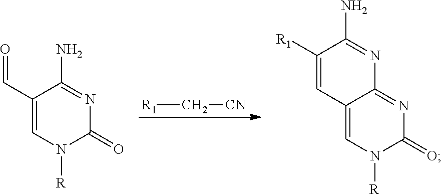

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl; sequencing the nucleic acid from the sample to obtain a test sequence wherein the adduct is read as thymine (T) during sequencing; and comparing the test sequence with a reference sequence, wherein a transition from a cytosine (C) in the reference sequence to a thymine (T) in the corresponding position in the test sequence indicates the presence of a methylated cytosine in the nucleic acid. In some embodiments, the compound of formula R₁—CH₂—CN is malononitrile. In some embodiments, the reaction mixture further contains one or more of the following: an organic acid, a non-aqueous solvent and a compound of formula R_{X}NH_{y} wherein x and y are 0, 1, 2 or 3 so that x+y=3, and each R is independently selected from C1-C6 linear or branched alkyl which may comprise heteroatoms such as O and N, C6-C10-aryl, or 5- or 6-membered heteroaryl. In some embodiments, the organic acid is acetic acid, the non-aqueous solvent is ethanol or methanol and the compound of formula RₓNH_{y} is tri-ethanolamine or piperidine. In some embodiments, the non-aqueous solvent is present in the reaction mixture at a concentration of 10-100%, e.g., 90-100%. In some embodiments, the reaction mixture is incubated for 1 hour. In some embodiments, prior to sequencing, the nucleic acid is amplified with a B-family polymerase efficiently incorporating an adenine (A) nucleotide opposite the adduct. In some embodiments, TET and laccase are active in the same reaction mixture. In other embodiments, TET and laccase are not active in the same reaction mixture and are added consecutively to the sample.

In some embodiments, the invention is a method of detecting 5-hydroxymethyl cytosine (5hmC) in nucleic acids comprising the steps of: contacting a sample comprising the nucleic acid comprising 5hmC with laccase under conditions suitable for oxidizing 5hmC into 5-formyl cytosine (5fC); contacting the sample with a Wittig reagent; irradiating the sample with an ultraviolet light to form a product; sequencing the nucleic acid from the reaction mixture to obtain a test sequence wherein the product is read as thymine (T) during sequencing; and comparing the test sequence with a reference sequence, wherein a transition from a cytosine (C) in the reference sequence to a thymine (T) in the corresponding position in the test sequence indicates the presence of 5hmC in the nucleic acid. In some embodiments, the 5fC conversion reagent is the compound of formula Ph₃P═C(R2)CN. In some embodiments, the compound is Ph₃P═C(CN)₂ (R2=—CN).

In some embodiments, the invention is a method of forming 5-formyl cytosine (5fC) in a nucleic acid comprising contacting a reaction mixture containing the nucleic acid including at least one 5-methyl cytosine (5mC) and/or 5-hydroxymethyl cytosine (5hmC) with an enzyme selected from xylene monooxygenase, toluene methyl-monooxygenase (EC 1.14.15.26), P450 monooxygenase (EC 1.14.14.1), alcohol dehydrogenase, alcohol oxidase, galactose oxidase, chloroperoxidase and peroxidase.

In some embodiments, the invention is a kit for detecting methylated cytosine in nucleic acids comprising laccase. In some embodiments, the laccase is isolated from a species selected from Hexagonia tenuis, Pleurotis sajor caju, Pleutoris ostreatus, Xylaria polymorpha, Trametes hirsuta, Trametes versicolor and Coprinus spp. In some embodiments, the laccase is isolated from a strain selected from Pleurotis sajor caju MTCC-141, Pleutoris ostreatus MTCC-1801, Xylaria polymorpha MTCC-1100, and Trametes hirsuta MTCC-1171. In some embodiments, the kit further comprises a laccase cofactor selected from 2,2,6,6-tetramethylpiperidine-1-oxyl (TEMPO), acetosyringone, syringaldehyde, para-coumaric acid 2,2′-azino-bis(3-ethylbenzothiazoline-6-sulfonate (ABTS), violuric acid (VLA), N-acetyl-N-phenylhydroxylamine (NHA), N-hydroxybenzotriazole (HBT), and N-hydroxyphthalimide (HPI). In some embodiments, the kit further comprises one or more of the following: nucleic acid sequencing reagents, nucleic acid amplification reagents, and nucleic acid purification reagents. In some embodiments, the kit further comprises a ten-eleven-translocation (TET) dioxygenase enzyme. In some embodiments, TET and laccase are present in the same tube. In some embodiments, the kit further comprises an ethanol solution of malononitrile. In some embodiments, the kit further comprises a solution of acetic acid. In some embodiments, the kit further comprises a solution of triethanolamine.

In some embodiments, the invention is a kit for detecting methylated cytosine in nucleic acids comprising an enzyme capable of converting 5mC into 5hmC and/or 5fC, the enzyme selected from xylene monooxygenase, toluene methyl-monooxygenase (EC 1.14.15.26) and P450 monooxygenase (EC 1.14.14.1). In some embodiments, the invention is a kit for detecting methylated cytosine in nucleic acids comprising an enzyme capable of converting 5hmC into 5fC, the enzyme selected from alcohol dehydrogenase, alcohol oxidase, galactose oxidase, chloroperoxidase and peroxidase.

In some embodiments, the invention is a method of detecting a hydroxymethylated cytosine (5hmC) nucleotide in a nucleic acid, the method comprising: (i) ligating adaptors to a nucleic acid comprising 5-hydroxymethyl cytosine (5hmC), wherein adaptors comprise amplification primer binding sites; (ii) forming a reaction mixture by contacting the sample containing adaptor-ligated nucleic acid with a laccase capable of converting 5hmC in the nucleic acid into 5-formyl cytosine (5fC); (iii) contacting the reaction mixture with malononitrile to form a 5fC adduct; (iv) amplifying the nucleic acids from step (iii) utilizing a DNA polymerase and primers capable of binding to the primer-binding sites, wherein the DNA polymerase reads the 5fC adduct as thymine (T) during amplification; (v) sequencing the amplified nucleic acid to obtain a test sequence; (vi) comparing the test sequence with a reference sequence, wherein a transition from a cytosine (C) in the reference sequence to a thymine (T) in the corresponding position in the test sequence indicates the presence of a hydroxymethylated cytosine in the nucleic acid.

In some embodiments, the invention is a method of detecting a methylated cytosine (5mC) nucleotide in a nucleic acid, the method comprising: (i) ligating adaptors to a nucleic acid comprising 5-methyl cytosine (5mC), wherein adaptors comprise amplification primer binding sites; (ii) forming a reaction mixture by contacting the sample containing adaptor-ligated nucleic acid with a TET enzyme capable of converting 5mC in the nucleic acid into 5hmC and laccase capable of converting 5hmC in the nucleic acid into 5-formyl cytosine (5fC); (iii) contacting the reaction mixture with malononitrile to form a 5fC adduct; (iv) amplifying the nucleic acids from step (iii) utilizing a DNA polymerase and primers capable of binding to the primer-binding sites, wherein the DNA polymerase reads the 5fC adduct as thymine (T) during amplification; (v) sequencing the amplified nucleic acid to obtain a test sequence; (vi) comparing the test sequence with a reference sequence, wherein a transition from a cytosine (C) in the reference sequence to a thymine (T) in the corresponding position in the test sequence indicates the presence of a methylated cytosine in the nucleic acid.

In some embodiments, the invention is a method of detecting a tissue of origin of a nucleic acid in a sample, the method comprising detecting the presence and location of methylated cytosines in the nucleic acid by the method as disclosed herein comparing the methylation pattern to the known methylation patterns of several tissues; and identifying the tissue of origin of the nucleic acid in the sample.

In some embodiments, the invention is a method of detecting organ transplant rejection in a transplant recipient, the method comprising obtaining from the transplant recipient a blood sample containing cell-free nucleic acids; detecting the presence and location of methylated cytosines in the cell-free nucleic acid by the method described herein, comparing the methylation pattern to the known methylation patterns of several organs; detecting transplant rejection if the cell-free nucleic acid with the transplanted organ-specific methylation pattern is detected in the sample.

In some embodiments, the invention is a method of monitoring for transplant rejection by periodically sampling circulating cell-free DNA and detecting the presence and location of methylated cytosines according to the method described herein, measuring changes in the level of cell-free DNA with the transplanted organ-specific methylation pattern, wherein an increase in the level of such cell-free DNA indicates organ transplant rejection.

In some embodiments, the invention is a method of screening for the presence of a cancerous tumor in a patient, the method comprising obtaining from the patient a blood sample containing cell-free nucleic acids; detecting the presence and location of methylated cytosines in the cell-free nucleic acids by the method described herein, comparing the methylation pattern to the known methylation patterns of tumor and non-tumor tissues, and detecting the presence of a tumor if tumor-specific methylation patter is detected.

In some embodiments, the invention is a method of monitoring tumor volume in a patient the method comprising periodically sampling circulating cell-free DNA and detecting the presence and location of methylated cytosines according to the method described herein, measuring changes in the level of cell-free DNA with the tumor-specific methylation pattern, wherein an increase in the level of such cell-free DNA indicates tumor growth, while a decrease in the level of such cell-free DNA indicates tumor shrinkage.

In some embodiments, the invention is a method of monitoring the effectiveness of treatment of cancer in a patient by a method comprising periodically sampling circulating cell-free DNA and detecting the presence and location of methylated cytosines according to the method described herein, measuring changes in the level of cell-free DNA with the tumor-specific methylation pattern, wherein an increase in the level of such cell-free DNA indicates that treatment ineffective, while a decrease in the level of such cell-free DNA indicates treatment effectiveness.

In some embodiments, the invention is a method of diagnosis or minimal residual disease (MRD) in a cancer patient the method comprising obtaining from the patient a blood sample comprising cell-free nucleic acids, detecting the presence and location of methylated cytosines in the nucleic acid by the method described herein, comparing the methylation pattern to the known methylation patterns of several tissues; and identifying the tissue of origin of the nucleic acid in the sample.

In some embodiments, the invention is a method of diagnosing an autoimmune disease in a patient the method comprising from the patient a blood sample comprising cell-free nucleic acids, detecting the presence and location of methylated cytosines in the nucleic acid by the method described herein, comparing the methylation pattern to the known methylation patterns of tissues damaged by the immune disease; diagnosing immune disease if such methylation pattern is found.

In some embodiments, the invention is a method of distinguishing 5-hydroxymethylcytosine (5hmC) from 5-methylcytosine (5mC) in nucleic acids in a sample, the method comprising: separating a sample into two aliquots; in the first aliquot, contacting the nucleic acid comprising 5mC and 5hmC with a ten-eleven-translocation (TET) dioxygenase under conditions where 5hmC and 5mC are converted into 5-formyl cytosine (5fC); in the second aliquot, contacting the nucleic acid comprising 5mC and 5hmC with a laccase under conditions where 5hmC is converted into 5-formyl cytosine (5fC); contacting both aliquots separately with a compound of formula R₁—CH₂—CN capable of reacting with 5fC in the nucleic acid to form an adduct according to the reaction scheme

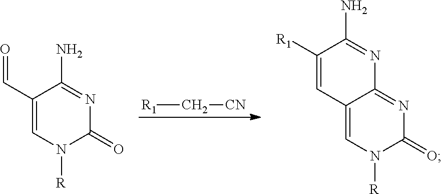

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl; sequencing the nucleic acid from both aliquots separately to obtain a first and a second test sequence wherein the adduct is read as thymine (T) during sequencing; and comparing the first test sequence with a reference sequence, wherein a transition from a cytosine (C) in the reference sequence to a thymine (T) in the corresponding position in the test sequence indicates the presence of 5hmC and 5mC in the nucleic acid; comparing the second test sequence with a reference sequence, wherein a transition from a cytosine (C) in the reference sequence to a thymine (T) in the corresponding position in the test sequence indicates the presence of 5hmC in the nucleic acid; comparing the first test sequence with a second test sequence, wherein only such cytosines of the first test sequence are 5mC which are not detected as hydroxymethylated cytosines (5hmC) in the second test sequence. Alternatively, it is possible to contact both aliquots separately with a Wittig reagent.

### BRIEF DESCRIPTION OF THE DRAWINGS

FIG. 1 shows DHU conversion using ethanol as co-solvent of picoline-borane in methylation detection assays (TAPS).

FIG. 2 shows DHU conversion using methanol and acetic acid as co-solvents of picoline-borane in methylation detection assays (TAPS).

FIG. 3 shows malononitrile adduct formation using malononitrile in a sodium acetate buffer in methylation detection assays.

FIG. 4 shows malononitrile adduct formation using malononitrile in an ethanol-TRIS buffer in methylation detection assays.

FIG. 5 shows malononitrile adduct formation using malononitrile in an ethanol-triethylamine buffer in methylation detection assays.

FIG. 6 shows conversion of CpG sites in a single-tube methylation detection assay with TET and malononitrile

FIG. 7 shows oxidation of 5hmC into 5fC in an oligonucleotide by laccase in the presence of TEMPO.

FIG. 8 shows that 5mC in the oligonucleotide is not oxidized by laccase under the same conditions.

FIG. 9A and FIG. 9B show Liquid Chromatography-Mass Spectrometry (LC-MS) data of how various amine buffer catalysts modulate TET activity to 5hmC and 5fC. In particular, FIG. 9A shows the effects of 2-Amino-5-methoxybenzoic acid, and FIG. 9B shows the effects of 2-(Aminomethyl)imidazole dihydrochloride on TET oxidation of 5mC to 5hmC/5fC.

FIG. 10 shows a table depicting the amounts of 5fC and 5caC produced by 5mC oxidation via laccase with TEMPO at two different temperatures, 25° C. and 37° C.

FIG. 11A shows LC-MS data of the effect of malononitrile on conversion of 5fC to 5fC-M adduct under various buffer conditions. The top graph of FIG. 11A shows buffer conditions of 40° C. for 1 hour, the middle graph of FIG. 11A shows buffer conditions of 60° C. for 1 hour, and the bottom graph of FIG. 11A shows buffer conditions of 95° C. for 10 minutes. FIG. 11B shows data showing the effect of pre-denaturation with NaOH on the activity of malononitrile.

FIG. 12 shows LC-MS data showing the oxidation of 5hmC in as little as 22 hours in Cu 2,2,6,6-tetramethylpiperidine-1-oxyl (CuTEMPO). The top graph of FIG. 12 shows the oxidation of 5hmC in CuTEMPO, and the bottom graph of FIG. 12 shows the derivatization of the products from the top graph with DMEAH.

FIG. 13A shows the reaction of 5fC-M adduct conversion to Thymine (T), which is mediated by the activity of polymerase enzymes. FIG. 13B shows the composition of the optimized buffer (“DOE_1”) for polymerases. FIG. 13C shows the conversion of 5fC-M adduct to T using standard buffer (“BufferA”) and an optimized buffer (“DOE_1”).

### DETAILED DESCRIPTION OF THE INVENTION

### Abbreviations

Some abbreviations used throughout this disclosure are listed below.
C—cytosine
T—thymine
U—uracil
DHU—dihydrouracil
5mC—5-methylcytosine
5hmC—5-hyrdoxymethyl cytosine
5ghmC—5-glucosyl-hydroxymethyl cytosine
5fC—5-formylcytosine
5caC—5-carboxycytosine
TET—ten-eleven translocation dioxygenase
TAPS—TET-assisted pic-borane sequencing
CAPS—chemically-assisted pic-borane sequencing
oxBS or oxBS-Seq—oxidative bisulfite sequencing

5-Methyl cytosine and 5-hydroxymethyl cytosine (5mC and 5hmC) are important epigenetic biomarkers with many clinical applications in oncology, prenatal testing and other fields. Until recently, base-level detection of methylation was achieved by reacting unmethylated cytosines with bisulfite followed by PCR, array hybridization or sequencing. Unmethylated cytosines (C) would read as thymine (T) after reacting with bisulfite, while methylated cytosines (5mC and 5hmC) would read as C. Unfortunately, bisulfite treatment leads to degradation of large portion of sample nucleic acid making it unsuitable for applications requiring high sensitivity. For example, the method is unsuitable for latest applications analyzing cell-free nucleic acid such as cell-free DNA.

Recently, less harsh methods for the detection of methylated cytosines have been disclosed. The newest methods involve modification of the methylated cytosines instead of the unmethylated cytosines, as is the case with bisulfite treatment Liu, Y., et al. (2019) Bisulfite-free direct detection of 5-methylcytosine and 5-hydroxymethylcytosine at base resolution. Nat Biotechnol. 37, 424-429. The stepwise oxidation of methyl-cytosines (5mC) via 5-hydroxymethyl cytosine (5hmC) to formyl cytosine (5fC) and carboxyl cytosine (5caC) is performed using ten-eleven translocation dioxygenases (TET) in the presence of Fe(II) ions and alpha-ketoglutarate.

Liu et al. further described reducing 5fC (and 5caC) using borane derivatives (such a pyridine borane, picoline borane and others) to dihydrouracil (DHU). DHU is then read by uracil-tolerant nucleic acid polymerases as T in subsequent amplification and sequencing. As a result, methylated C is read as T, while unmethylated C is unchanged. This TET and picoline-borane based method called TAPS (TET-assisted picoline-borane sequencing) does not cause DNA degradation as much as bisulfite treatment and allows detection of the signal directly instead of subtracting background to obtain signal. Both advantages would allow higher alignment rates, possibly lower sequencing depth and recover higher molecular diversity from the sample.

Another technique termed CAPS (Chemically Assisted Picoline-borane Sequencing) involves the selective conversion of 5hmC to 5fC using potassium perruthenate (KRuO₄). The use of KRuO₄ as a chemical alternative to TET is known from a technique termed Oxidative Bisulfite Sequencing or oxBS-seq, see Booth M. J., et al. (2012) Quantitative sequencing of 5-methylcytosine and 5-hydroxymethylcytosine at single base resolution, Science 12 May: 934-937. The 5fC obtained by potassium perruthenate conversion becomes a favorable target for further processing by e.g. borane treatment or any other downstream method.

Yet another sequencing technique is an alternative to the reduction of 5fC with borane. This method involves forming an adduct of 5fC recognized as T. The adduct is formed with the use of malononitrile, see Zhu C., et al., (2017) Single-Cell 5-Formylcytosine Landscapes of Mammalian Early Embryos and ESCs at Single-Base Resolution, Cell Stem Cell, 20:720-731.e5.

TAPS, CAPS and the malononitrile method of Zhu et al. are superior to bisulfite method in that they avoid the harsh chemical treatment and the resulting loss of sample nucleic acids. However, the newer methods have a disadvantage of taking a very long time to complete or require high temperatures: 3 h at 70° C. or 16 h at 37° C. for borane reactions of TAPS (see Liu et al., Nature Biotech. 37, pages 424-429(2019) or 1-2 days to form the malononitrile adduct (see U.S. Pat. No. 10,519,184 and application Pub. No. US20200165661). The instant disclosure comprises improved and more practical methods of detecting cytosine methylation in nucleic acids.

The various aspects of the invention are described in further detail below.

In some embodiments, the invention is a method of detecting an epigenetic modification, specifically, cytosine methylation in nucleic acids. The state of the art methods of detecting methylated cytosines in nucleic acids include the following key steps: 1) oxidation of methylated cytosine; 2) reduction of the oxidized product into a form capable of being read as thymine (T) during sequencing; 3) sequencing the nucleic acids; and 4) comparing the treated and untreated sequences wherein a change from a cytosine (T) to a thymine (T) in the sequence read indicated the presence of a methylated cytosine. The instant invention comprises several useful improvements to the general scheme set forth above.

In some embodiments, the invention is a method comprising an improved step 1) oxidizing methylated cytosine, and steps 2)-4) performed according to the state of the art. In some embodiments, the invention is a method comprising an improved step 2) reduction of the oxidized product, and steps 1), 3) and 4) performed according to the state of the art. In some embodiments, the invention is a method comprising an improved step 1) oxidizing methylated cytosine, an improved step 2) reduction of the oxidized product, and steps 3) and 4) performed according to the state of the art.

The present invention involves a method of manipulating nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood or blood fractions, i.e., plasma, lymph, saliva, sputum, sweat, tear, cerebrospinal fluid, amniotic fluid, synovialfluid, pericardialfluid, peritonealfluid, pleuralfluid, cystic fluid, bile, gastric fluid, intestinal fluid, or fecal samples). In other embodiments, the sample is a cultured sample, e.g., a tissue culture containing cells and fluids from which nucleic acids may be isolated. In some embodiments, the nucleic acids of interest in the sample come from infectious agents such as viruses, bacteria, protozoa or fungi.

The present invention involves manipulating isolated nucleic acids isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of kits are commercially available for extracting nucleic acids (DNA or RNA) from biological samples (e.g., KAPA Express Extract (Roche Sequencing Solutions, Pleasanton, Cal.) and other similar products from BD Biosciences Clontech (Palo Alto, Cal.), Epicentre Technologies (Madison, Wisc.); Gentra Systems, (Minneapolis, Minn.); and Qiagen (Valencia, Cal.), Ambion (Austin, Tex.); BioRad Laboratories (Hercules, Cal.); and more.

In some embodiments, nucleic acids are extracted, separated by size and optionally, concentrated by epitachophoresis as described e.g., in WO2019092269 and WO2020074742.

The present invention involves detecting epigenetic modification in nucleic acids. The nucleic acid sequences that are subject to conditional epigenetic modification are the target sequences analyzed by the method disclosed herein. The same nucleic acid sequence may or may not have the epigenetic modification characterized by methylation of cytosines at the 5-position (5mC or 5hmC). In some embodiments, a set or a panel of target nucleic acids are probed for the presence of methylation. For example, as shown in Patai A V, et al. (2015) Comprehensive DNA Methylation Analysis Reveals a Common Ten-Gene Methylation Signature in Colorectal Adenomas and Carcinomas. PLOS ONE 10(8): e0133836 and in Onwuka, J. U., et al. (2020) A panel of DNA methylation signature from peripheral blood may predict colorectal cancer susceptibility. BMC Cancer 20, 692, methylation of biomarkers in a panel of methylation biomarkers is indicative of the presence of colorectal cancer in the patient. Accordingly, testing any known or future panels of methylation biomarkers for prognostic or diagnostic purposes is envisioned with the method disclosed herein.

In some embodiments, the entire genome of an organism is probed for the presence of methylation. The method of the instant invention includes detecting methylation in all sites throughout the genome of an organism to diagnose a disease or condition or predisposition to a disease or condition using the sequence analysis and artificial intelligence tools described e.g., in Shull A Y, et al., (2015) Sequencing the cancer methylome. Methods Mol Biol. 1238:627-5.

In some embodiments, it is desired to separately detect or distinguish 5mC and 5hmC in a sample. In this embodiment, 5hmC is blocked from oxidation and is not converted to a compound read as T during sequencing. The blocking process takes advantage of the reactive hydroxyl group present on 5hmC but not 5mC. In some embodiments, the blocking group added to 5hmC is a sugar moiety. In some embodiments, the sugar moiety is a modified or unmodified glucose moiety and 5-glucosyl-hydroxymethyl cytosine (5ghmC) is formed. 5ghmC does not undergo adduct formation or reduction with borane derivatives according to the scheme known for 5fC and 5hmC. In some embodiments, addition of the blocking group is catalyzed by a glycosyltransferase, e.g., a glucosyltransferase. In some embodiments, 5hmC in nucleic acid is reacted with a modified glucose in the presence of a beta-glucosyltransferase. In some embodiments, the modified glucose is UDP-glucose and the catalyst is a bacteriophage T4 beta-glucosyltransferase (T4 BGT).

In some embodiments, the method includes a step of oxidizing methylated cytosines for downstream detection. In some embodiments, the method includes a step of converting 5-methyl cytosine (5mC) and/or 5-hydroxymethyl cytosine (5hmC) into 5-formyl cytosine (5fC) or 5-carboxyl cytosine (5caC) or a mixture of 5fC and 5caC. In some embodiments, the invention comprises a step of contacting a sample or a reaction mixture with a ten-eleven-translocation (TET) dioxygenase as described e.g., in the U.S. Pat. No. 9,115,386 or U.S. Application Pub. No. US20200370114. In some embodiments, the TET enzyme is selected from TET1, TET2, TET3 and a related protein CXXC4. In some embodiments, TET is selected from mouse TET1, TET2 or TET3 (mTET1, 2 or 3), human TET1, TET2 or TET3 (hTET1, 2 or 3), Naegleria TET (NgTET), Coprinopsis cinerea (CcTET) or any other analog or equivalent thereof with similar or equivalent enzymatic activity.

In some embodiments, the invention utilizes a step of converting 5mC and/or 5hmC in the sample into predominantly or exclusively 5fC. In some embodiments, the invention utilizes a step of converting 5mC and 5hmC in the sample into predominantly or exclusively 5caC. In some embodiments, the invention utilizes a step of converting 5mC and 5hmC in the sample into a mixture of 5fC and 5caC.

In some embodiments, the invention comprises a step of converting 5mC and 5hmC in the sample into predominantly or exclusively 5fC by contacting the sample with TET in the presence of Fe(II) ions. In some embodiments a suitable source or Fe(II) ions is selected from example FeSO₄, (NH₄)₂Fe(SO₄)₂, FeSO₄7H₂O, (NH₄)₂Fe(SO₄)₂6H₂O, FeCl₂ and similar examples. In some embodiments, the invention comprises a step of converting 5mC and 5hmC in the sample into 5fC by contacting the sample with TET and 5-10 μM of a Fe(II) ions at pH 7-8. In some embodiments, the invention utilizes a step of converting 5mC and 5hmC in the sample into 5fC by incubation with TET and 5-1 μM of a Fe(II) ions at pH 8. In some embodiments, the invention utilizes a step of converting 5mC and 5hmC in the sample into 5fC by incubation with TET and 80-10 μM of a Fe(II) ions at pH 7.

In some embodiments, the invention comprises a step of converting 5mC and 5hmC in the sample into predominantly or exclusively 5fC by contacting the sample with TET and Fe(II) ions in the presence of ascorbic acid, alpha-ketoglutarate and a reducing agent.

In some embodiments, the invention utilizes a step of converting 5hmC in the sample into predominantly or exclusively 5fC by contacting the sample with Cu(II) compound and 2,2,6,6-tetramethylpiperidine-1-oxyl (TEMPO).

In some embodiments, the invention comprises improved steps of detecting methylated cytosines in nucleic acids by detecting 5-formyl cytosine (5fC) nucleotide in a nucleic acid, wherein the 5fC is formed by one of the methods described herein above. The method involves contacting a sample containing a nucleic acid comprising 5fC with an improved composition comprising a compound of formula R₁—CH₂—CN in improved solvent composition, the compound being capable of reacting with 5fC in the nucleic acid to form an adduct according to the reaction scheme

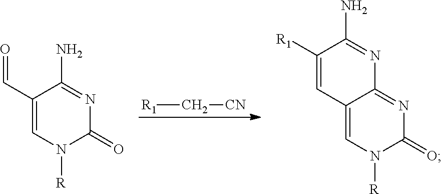

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl, or heteroaryl. The reactants of the above reaction are described e.g., in U.S. Pat. No. 10,519,184 and application Pub. No. US20200165661 by Yi et al. For example, R1 is a cyano group (CN) and the reactant is malononitrile.

The instant invention provides an improved composition of the reaction mixture which improves on the Yi method by enabling the reaction to proceed for less than 3 hours wherein at least 90% of 5fC has formed the adduct. In some embodiments, the reaction proceeds for only 1 hour with at least 90% of 5fC forming the adduct. By contrast, the Yi reaction requires no less than 20 hours and up to 48 hours (see US20200165661, Examples).

In some embodiments, the improvement over the prior art involves conducting the reaction between 5fC and the compound of formula R₁—CH₂—CN in a solution comprising an organic acid moiety. The organic acid has a formula R—COOH and R is selected from C1-C30 linear or branched alkyl, C1-C30 linear or branched alkenyl, C1-C30 linear or branched alkynyl, cycloalkyl, aryl or heteroaryl. In some embodiments, the reaction takes place in the presence of acetic acid. In some embodiments, the concentration of the organic acid in the reaction is between 1% and 30%, e.g., 1%, 2%, 3%, 4%, 5%, 6%, 7%, 8%, 9%, 10%, 11%, 12%, 13%, 14%, 15%, 16%, 17%, 18%, 18%, 19%, 20%, 21%, 22%, 23%, 24%, 25%, 26%, 27%, 28%, 29%, or 30%.

In some embodiments, the improvement over the prior art involves conducting the reaction between 5fC and the compound of formula R₁—CH₂—CN in a non-aqueous solvent. The non-aqueous solvent has a formula R—OH wherein R is selected from C1-C30 linear or branched alkyl, C1-C30 linear or branched alkenyl, C1-C30 linear or branched alkynyl, cycloalkyl, aryl or heteroaryl. In some embodiments, the reaction takes place in methanol or ethanol. In some embodiments, the reaction takes place in 10%-100% methanol or ethanol. In some embodiments, the reaction takes place in 90% or more of methanol or ethanol.

In some embodiments, the improvement over the prior art involves conducting the reaction between 5fC and the compound of formula R₁—CH₂—CN in a solution comprising a compound of formula RₓNH_{y} wherein x and y are 0, 1, 2 or 3 so that x+y=3, and each R is independently selected from C1-C30 linear or branched alkyl, C1-C30 linear or branched alkenyl, C1-C30 linear or branched alkynyl, cycloalkyl, aryl or heteroaryl. In some embodiments, the compound of formula RₓNH_{y} is a primary, secondary, or tertiary amine with aliphatic or aromatic groups. In some embodiments, reaction takes place in the presence of triethanolamine.

In some embodiments, the improvement over the prior art involves conducting the reaction between 5fC and the compound of formula R₁—CH₂—CN simultaneously with TET oxidation to enable a simplified, one-tube workflow. In some embodiments, TET and malononitrile are added simultaneously and oxidation to 5fC and 5fC-malononitrile adduct formation take place in the same tube.

In some embodiments, the invention comprises improved steps of detecting methylated cytosine in nucleic acids by forming and detecting 5-carboxylcytosine (5caC) and 5-formylcytosine (5fC), wherein 5fC, 5caC or a mixture of 5fC and 5caC are formed by one of the methods described herein above. The method involves contacting a sample containing a nucleic acid comprising 5fC and/or 5caC with an improved composition comprising a borane derivative in improved solvent composition, the borane derivative being capable of reacting with 5caC and with lesser efficiency with 5fC in the nucleic acid to form dihydrouracil (DHU). Examples of borane derivatives include 2-picoline borane (pic-borane), pyridine borane, tert-butylamine borane, ethylenediamine borane and dimethylamine borane as described by Song and Liu in WO2019136413 (TET-Assisted Picoline borane Sequencing or TAPS).

The instant invention provides an improved composition of the borane-containing reaction mixture, which improves on the TAPS method by enabling the reaction to proceed for less than one hour at 35° C. wherein nearly all of 5caC is converted to DHU. In some embodiments, the reaction proceeds for only ½ hour with nearly all of 5caC being converted. By contrast, the TAPS borane reaction as described by Liu et al., requires no less than 3 hours at 70° C. or 16 hours and 37° C. (see WO2019136413, Examples: Borane Reduction).

In some embodiments, the improvement over the prior art involves conducting the reaction between 5fC or 5caC and the borane derivative in a solution comprising an organic acid moiety. The organic acid has a formula R—COOH and R is selected from C1-C30 linear or branched alkyl, C1-C30 linear or branched alkenyl, C1-C30 linear or branched alkynyl, cycloalkyl, aryl or heteroaryl. In some embodiments, the reaction takes place in the presence of acetic acid.

In some embodiments, the improvement over the prior art involves conducting the reaction between 5fC or 5caC and the borane derivative in a non-aqueous solvent. The non-aqueous solvent has a formula R—OH wherein R is selected from C1-C30 linear or branched alkyl, C1-C30 linear or branched alkenyl, C1-C30 linear or branched alkynyl, cycloalkyl, aryl or heteroaryl. In some embodiments, the reaction takes place in methanol or ethanol. In some embodiments, the reaction takes place in 90% or more of methanol or ethanol.

Following the formation of the adduct (in case of the malononitrile treatment) or DHU (in case of the borane treatment), the nucleic acid with the adduct or with DHU is subjected to sequencing. In some embodiments, sequencing is by a next-generation massively parallel sequencing process. Sequencing results in a test sequence wherein the adduct or DHU are read as thymine (T), i.e., the sequencing polymerase is able to accommodate the adduct or DHU in the strand being copied, and to incorporate an adenine (A) opposite the adduct or DHU. The method further comprises a step of comparing the test sequence with a reference sequence, wherein a change from a cytosine (C) in the reference sequence to a thymine (T) in the corresponding position in the test sequence indicates the presence of methylated cytosine in the test nucleic acid.

In some embodiments, the nucleic acids in the sample are amplified prior to sequencing. In some embodiments, amplification utilizes a B-family polymerase efficiently incorporating an adenine (A) nucleotide opposite the malononitrile adduct or DHU. In this embodiment, the sequencing may proceed with any polymerase suitable for the sequencing process as the adduct or DHU have already been recognized as T by the amplification polymerase.

In some embodiments, the nucleic acid in the sample is ligated to adaptors, wherein adaptors comprise elements useful in amplification and sequencing. An adaptor comprises at least one of the following: barcode, primer binding site and ligation site.

In some embodiments, the invention is an improved method of detecting a methylated cytosine nucleotide in a nucleic acid, the method comprising: (i) ligating adaptors to a nucleic acid in a sample wherein adaptors comprise amplification primer binding sites; (ii) forming a reaction mixture by contacting the sample containing adaptor-ligated nucleic acid with TET capable of converting 5mC in the nucleic acid into 5-formyl cytosine (5fC); (iii) contacting the reaction mixture with a compound of formula R₁—CH₂—CN capable of reacting with 5fC in the nucleic acid to form an adduct according to the reaction scheme

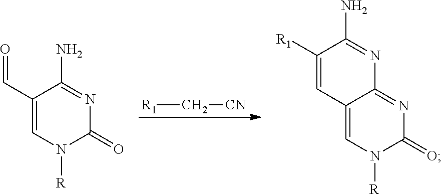

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl or heteroaryl (for example, a malononitrile); (iv) incubating the reaction mixture for less than 3 hours wherein at least 90% of 5fC has formed the adduct; (v) amplifying the adapted nucleic acids utilizing a DNA polymerase and primers capable of binding to the primer-binding sites, wherein the DNA polymerase reads the adduct as thymine (T) during amplification; (vi) sequencing the amplified nucleic acid to obtain a test sequence; (vii) comparing the test sequence with a reference sequence, wherein a change from a cytosine (C) in the reference sequence to a thymine (T) in the corresponding position in the test sequence indicates the presence of 5mC in the nucleic acid. In some embodiments, in step (iii) the compound of formula R₁—CH₂—CN is present in a non-aqueous solvent, e.g., ethanol or methanol. In some embodiments, in step (iii) the compound of formula R₁—CH₂—CN is present in a solution comprising an organic acid such acetic acid. In some embodiments, in step (iii) the compound of formula R₁—CH₂—CN is present in a solution comprising an amine such as triethanolamine.

In some embodiments, the invention is an improved method of detecting a methylated cytosine nucleotide in a nucleic acid, the method comprising: (i) ligating adaptors to a nucleic acid in a sample wherein adaptors comprise amplification primer binding sites; (ii) forming a reaction mixture by contacting the sample containing adaptor-ligated nucleic acid with TET capable of converting the methylated cytosine in the nucleic acid into 5-carboxycytosine (5caC) or a mixture of 5-formyl cytosine (5fC) and 5caC; (iii) contacting the reaction mixture with a borane derivative capable of reacting with 5fC and 5caC in the nucleic acid to form DHU (iv) incubating the reaction mixture for no more than about 1 hour wherein at least 90% of 5fC and 5caC has formed DHU; (v) amplifying the adapted nucleic acids utilizing a DNA polymerase and primers capable of binding to the primer-binding sites, wherein the DNA polymerase reads DHU as thymine (T) during amplification; (vi) sequencing the amplified nucleic acid to obtain a test sequence; (vii) comparing the test sequence with a reference sequence, wherein a change from a cytosine (C) in the reference sequence to a thymine (T) in the corresponding position in the test sequence indicates the presence of 5mC in the nucleic acid. In some embodiments, in steps (iii) and (iv) the borane derivative is present in a non-aqueous solvent, e.g., ethanol or methanol. In some embodiments, in steps (iii) and (iv) the borane derivative is present in a solution comprising an organic acid such acetic acid.

In some embodiments, the invention is a single-tube method of detecting methylation in nucleic acids. In some embodiments, the method comprises: (i) ligating adaptors to a nucleic acid in a sample wherein adaptors comprise amplification primer binding sites; (ii) forming a reaction mixture by contacting the sample containing adaptor-ligated nucleic acid with TET capable of converting 5mC in the nucleic acid into 5-formyl cytosine (5fC) or a mixture of 5-carboxy cytosine (5caC) and 5fC and simultaneously, contacting the same reaction mixture with a compound of formula R₁—CH₂—CN capable of reacting with 5fC in the nucleic acid to form an adduct according to the reaction scheme

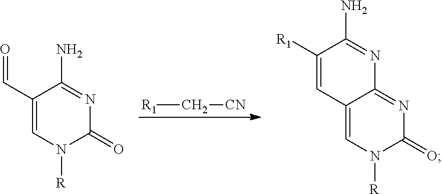

wherein, R₁ is an electron-withdrawing group selected from substituted or unsubstituted cyano, nitro, formyl, carbonyl compound, wherein the substitution is selected from C1-C30 linear or branched alkyl, C1-C30 linear or branched alkenyl, C1-C30 linear or branched alkynyl, cycloalkyl, aryl or heteroaryl (for example, a malononitrile); (iu) incubating the reaction mixture to enable 5fC to form the adduct; (iv) amplifying the adapted nucleic acids utilizing a DNA polymerase and primers capable of binding to the primer-binding sites, wherein the DNA polymerase reads the adduct as thymine (T) during amplification; (v) sequencing the amplified nucleic acid to obtain a test sequence; (vi) comparing the test sequence with a reference sequence, wherein a change from a cytosine (C) in the reference sequence to a thymine (T) in the corresponding position in the test sequence indicates the presence of 5mC in the nucleic acid. In some embodiments, in step (ii) the conditions are optimized to improve performance of TET. In some embodiments, in steps (ii) and (iii) the reaction mixture comprises a non-aqueous solvent, e.g., ethanol or methanol. In some embodiments, in steps (ii) and (iii) the reaction mixture comprises an organic acid such acetic acid. In some embodiments, in steps (ii) and (iii) the reaction mixture comprises an organic amine such triethanolamine.

In some embodiments, the invention includes a step of amplifying nucleic acids. In some embodiments, amplification occurs prior to the sequencing step. In some embodiments, amplification occurs after the step of forming an adduct of 5fC and malononitrile. In some embodiments, amplification occurs after the step of reduction of oxidized methylated cytosine with a borane derivative. In some embodiments, amplification occurs prior to the target enrichment step. The amplification utilizes an upstream primer and a downstream primer. In some embodiments, both primers are target specific primers, i.e., primers comprising a sequence complementary to the target sequence of the methylation biomarker. In other embodiments, one or both primers are universal primers. In some embodiments, universal primer binding sites are present in adaptors ligated to the target sequenced as described herein. In some embodiments, a universal primer binding site is present in the 5′-region (tail) of a target-specific primer. Accordingly, after one or more rounds of primer extension with a tailed target-specific primer, a universal primer may be used for subsequent rounds of amplification. In some embodiments, a universal primer in paired with another universal primer (of the same or different sequence). In other embodiments, a universal primer is paired with a target-specific primer.

In some embodiments, the invention involves a nucleic acid polymerase. Nucleic acid polymerases used in amplification and sequencing are known and commercially available from multiple sources. In some embodiments, the instant invention involves copying a strand comprising a 5fC adduct formed as described herein. Such copying requires a polymerase accommodating the 5fC adduct. In some embodiments, the polymerase is a B-family polymerase. In some embodiments, the polymerases is able to copy a strand comprising a 5fC adduct by recognizing the adduct as T (i.e., incorporating an A opposite the adduct). Polymerases able to accommodate the 5fC adduct described herein include DNA polymerases known to accommodate uracil (U) in a DNA strand. The polymerase may be a naturally-occurring or an engineered polymerase. In some embodiments, the polymerase is isolated from hyperthermophilic archaea e.g., genus Pyrococcus (e.g., Pyrococcus furious) or genus Thermus (e.g., Thermus aquaticus). In some embodiments, the polymerase is isolated from mesophilic archaea, e.g., genus Metanosarcina (e.g., Methanosarcina acetivorans). Examples of engineered uracil-tolerant polymerases include KAPA HiFi Uracil+ DNA polymerase (Roche Sequencing Solutions, Pleasanton, Cal.), Takara Terra (Takara Bio USA, Mountain View, Cal.), and EpiMark© Hot Start Taq DNA polymerase (New England Biolabs, Waltham, Mass.).

In some embodiments, the DNA polymerase is a type A DNA polymerase (DNA-dependent DNA polymerase). Some DNA polymerases possess limited terminal transferase activity (Taq polymerase adding a single dA at the 3′-end of the copy strand). Other DNA polymerases do not possess detectable terminal transferase activity. In such embodiments, a separate terminal transferase enzyme is used to add non-templated nucleotides to the 3′-end of the copy strand.

In some embodiments, the DNA polymerase is a Hot Start polymerase or a similar conditionally activated polymerase. For the amplification step, a thermostable DNA polymerase is used, for example polymerase is a Taq or Taq-derived polymerase (e.g., KAPA 2G polymerase from KAPA Biosystems, Wilmington, Mass.).

In some embodiments, the invention utilizes an adaptor added to one or both ends of a nucleic acid or nucleic acid strand. Adaptors of various shapes and functions are known in the art (see e.g., PCT/EP2019/05515 filed on Feb. 28, 2019, U.S. Pat. Nos. 8,822,150 and 8,455,193). In some embodiments, the function of an adaptor is to introduce desired elements into a nucleic acid. The adaptor-borne elements include at least one of nucleic acid barcode, primer binding site or a ligation-enabling site.

The adaptor may be double-stranded, partially single stranded or single stranded. In some embodiments, a Y-shaped, a hairpin adaptor or a stem-loop adaptor is used wherein the double-stranded portion of the adaptor is ligated to the double stranded nucleic acid formed as described herein.

In some embodiments, the adaptor molecules are in vitro synthesized artificial sequences. In other embodiments, the adaptor molecules are in vitro synthesized naturally-occurring sequences. In yet other embodiments, the adaptor molecules are isolated naturally occurring molecules or isolated non naturally-occurring molecules.

The double-stranded or partially double-stranded adaptor oligonucleotide can have overhangs or blunt ends. In some embodiments, the double-stranded DNA may comprise blunt ends to which a blunt-end ligation can be applied to ligate a blunt-ended adaptor. In other embodiments, the blunt ended DNA undergoes A-tailing where a single A nucleotide is added to the blunt ends to match an adaptor designed to have a single T nucleotide extending from the blunt end to facilitate ligation between the DNA and the adaptor. Commercially available kits for performing adaptor ligation include AVENIO ctDNA Library Prep Kit or KAPA HyperPrep and HyperPlus kits (Roche Sequencing Solutions, Pleasanton, CA). In some embodiments, the adaptor ligated (adapted) DNA may be separated from excess adaptors and unligated DNA.

In some embodiments, the invention includes the use of a barcode. In some embodiments, the method of detecting epigenetic modifications includes sequencing. The nucleic acid processed as described herein is subjected to sequencing; preferably, massively parallel single molecule sequencing. Analyzing individual molecules by massively parallel sequencing typically requires a separate level of barcoding for sample identification and error correction. The use of molecular barcodes such as described in U.S. Pat. Nos. 7,393,665, 8,168,385, 8,481,292, 8,685,678, and 8,722,368. A unique molecular barcode is added to each molecule to be sequenced to mark molecule and its progeny (e.g., the original molecule and its amplicons generated by PCR). The unique molecular barcode (UID) has multiple uses including counting the number of original target molecules in the sample and error correction (Newman, A., et al., (2014) An ultrasensitive method for quantitating circulating tumor DNA with broad patient coverage, Nature Medicine doi:10.1038/nm.3519).

In some embodiments, unique molecular barcodes (UIDs) are used for sequencing error correction. The entire progeny of a single target molecule is marked with the same barcode and forms a barcoded family. A variation in the sequence not shared by all members of the barcoded family is discarded as an artefact. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments of the invention, the adaptor ligated to one or both ends of the barcoded target nucleic acid comprises one or more barcodes used in sequencing. A barcode can be a UID or a multiplex sample ID (MID or SID) used to identify the source of the sample where samples are mixed (multiplexed). The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. In some embodiments, the number of UIDs in the reaction can be in excess of the number of molecules to be labelled. A person of ordinary skill would recognize that the number of barcodes depends on the complexity of the sample (i.e., expected number of unique target molecules) and would be able to create a suitable number of barcodes for each experiment.

In some embodiments, the method involves forming a library comprising nucleic acids from a sample. The library consists of a plurality of nucleic acids ready for sequencing or another type of detection method, e.g., PCR. A library can be stored and used multiple times for further processing such as amplification or sequencing of the nucleic acids in the library. In some embodiments, the library is the input nucleic acid in which methylation is detected by the method described herein. In other embodiments, the library is formed from nucleic acids that have undergone the methylation detection reactions described herein.

In some embodiments, the nucleic acids processed for detection of epigenetic modifications according to the method described herein are sequenced. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (U.S. Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeq™, MiSeq™, or Genome Analyzer, each available from Ilumina), sequencing by ligation (e.g., SOLiD™, Life Technologies), ion semiconductor sequencing (e.g., Ion Torrent™, Life Technologies), and SMRT© sequencing (e.g., Pacific Biosciences).

Commercially available sequencing technologies include: sequencing-by-hybridization platforms from Affymetrix Inc. (Sunnyvale, Calif.), sequencing-by-synthesis platforms from Illumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology from Roche Sequencing Solutions, Santa Clara, Cal.), and Oxford Nanopore Technologies (Oxford, UK).

In some embodiments, the sequencing step involves sequence aligning. In some embodiments, aligning is used to determine a consensus sequence from a plurality of sequences, e.g., a plurality having the same unique molecular ID (UID). The molecular ID is a barcode that can be added to each molecule prior to sequencing or if amplification step is included, prior to the amplification step. In some embodiments, a UID is present in the 5′-portion of the RT primer. Similarly, a UID can be present in the 5′-end of the last barcode subunit to be added to the compound barcode. In other embodiments, a UID is present in an adaptor and is added to one or both ends of the target nucleic acid by ligation.

In some embodiments, a consensus sequence is determined from a plurality of sequences all having an identical UID. The sequenced having an identical UID are presumed to derive from the same original molecule through amplification. In other embodiments, UID is used to eliminate artifacts, i.e., variations existing in the progeny of a single molecule (characterized by a particular UID). Such artifacts resulting from PCR errors or sequencing errors can be eliminated using UIDs.

In some embodiments, the number of each sequence in the sample can be quantified by quantifying relative numbers of sequences with each UID among the population having the same multiplex sample ID (MID). Each UID represents a single molecule in the original sample and counting different UIDs associated with each sequence variant can determine the fraction of each sequence variant in the original sample, where all molecules share the same MID. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the invention is a kit including components and tools for performing an improved method of detecting DNA methylation described herein. In some embodiments, the kit includes components for detecting cytosine methylation in nucleic acids by detecting a product of in vitro oxidized 5-methyl cytosine (5mC) or 5-hydroxymethyl cytosine (5hmC). In some embodiments, the product is 5-formyl cytosine (5fC) or 5-carboxy cytosine (5caC). In other embodiments, the kit further includes components for performing in vitro oxidation of 5-methyl cytosine (5mC) or 5-hydroxymethyl cytosine (5hmC) to 5-formyl cytosine (5fC) or 5-carboxy cytosine (5caC).

In some embodiments, the kit includes a borane derivative and a non-aqueous solvent. The borane derivative is selected from pyridine borane, 2-picoline borane (pic-BH3), borane, sodium borohydride, sodium cyanoborohydride, and sodium triacetoxyborohydride, while the non-aqueous solvent is selected from ethanol and methanol. In other embodiments, instead of including the non-aqueous solvent, the kit includes the borane derivative and instructions on using the non-aqueous solvent (such as ethanol or methanol) with the borane derivative in a method of detecting DNA methylation as described herein. In some embodiments, the kit further includes an organic acid. In some embodiments, the kid includes instructions on using the organic acid (such as acetic acid) in a method of detecting DNA methylation including borane derivatives in a non-aqueous solvent as described herein. In some embodiments, the kit further includes a buffer such as MES or TRIS.

In some embodiments, the kit includes malononitrile and a non-aqueous solvent. The non-aqueous solvent is selected from ethanol and methanol. In other embodiments, instead of including the non-aqueous solvent, the kit includes instructions on using the non-aqueous solvent (such as ethanol or methanol) in a method of detecting DNA methylation with malononitrile as described herein. In some embodiments, the kit further comprises an organic acid and a primary, a secondary or a tertiary amine. The organic acid may be acetic acid and the amine may be triethanolamine. In other embodiments, the kit includes instructions on using the organic acid and the amine (such as acetic acid and triethanolamine) in a method of detecting DNA methylation with malononitrile as described herein. In some embodiments, the kit further includes a buffer such as MES or TRIS.

In some embodiments, the kit further includes TET enzyme for in vitro oxidation of 5-methyl cytosine (5mC) or 5-hydroxymethyl cytosine (5hmC) to 5-carboxy cytosine (5caC). In some embodiments, TET is selected from mouse TET1, TET2 or TET3 (mTET1, 2 or 3), human TET1, TET2 or TET3 (hTET1, 2 or 3), Naegleria TET (NgTET), Coprinopsis cinerea (CcTET). In some embodiments, TET is Naegleria TET-like oxygenase (NgTET1). In some embodiments, TET is a wild-type protein. In other embodiments, TET is a mutant protein. In some embodiments, the kit further includes one or more co-factors selected from alpha-ketoglutarate and a source of Fe(II) ions.

In some embodiments, as an alternative to TET, the kit includes a chemical oxidative agent is included, e.g., potassium perruthenate (KRuO₄) or potassium ruthenate (K₂RuO₄).

In some embodiments, the kit further includes reagents for chemically blocking 5hmC from undergoing reactions that include 5mC. In some embodiments, the kit includes a glucose compound and a glucosyltransferase capable of transferring the glucose moiety to the 5-hydroxyl moiety of 5hmC. In some embodiments, the kit includes a beta-glucosyltransferase (BGT) and a UDP-glucose. In some embodiments, the BGT is T4 BGT.

In some embodiments, the method further comprises assessment of a status of a subject (e.g., a patient) based on the methylation status of one or more genetic loci in the patient's genome. In some embodiments, the method comprises determining in the patient's sample, the genomic location and optionally, amount of methylated cytosines (5mC and/or 5hmC) in the genome. In some embodiments, genetic loci known to be biomarkers of disease are assessed for methylation. The method further comprises diagnosis of disease or condition in the patient or selecting or changing a treatment based on the presence or amount of methylation in the nucleic acid isolated from the patient.

Several methods exists for identifying disease or condition-specific methylation loci that can be assessed for methylation using the methods disclosed herein, see e.g., US20200385813 “Systems and methods for estimating cell source fractions using methylation information;” US20200239965 “Source of origin deconvolution based on methylation fragments in cell-free DNA samples;” US20190287652 “Anomalous fragment detection and classification” (methylation markers indicating disease state); US20190316209 “Multi-assay prediction model for cancer detection;” US20190390257A1 “Tissue-specific methylation marker;” WO2011/070441 “Categorization of DNA samples;” WO2011/101728 “Identification of source of DNA samples;” WO2020/188561 “Methods and systems for detecting methylation changes in DNA samples.”

In some embodiments, the invention includes a method of detecting tissue-specific DNA methylation patterns using the methylation detection methods disclosed herein. In one aspect of this embodiment, the method may further include identifying a tissue of origin of the methylated DNA present in the sample. In some embodiments, the method further includes identifying a tissue of origin of cell-free DNA isolated from blood. In another aspect of this embodiment, the invention includes detection of organ failure or organ injury, including organ transplant rejection in a transplant recipient using methylation patterns of cell-free DNA. The invention includes detecting circulating cell-free DNA with the organ-specific methylation pattern, wherein the presence of such cell-free DNA indicates organ transplant rejection. In some embodiments, the invention includes monitoring for transplant rejection by periodically sampling circulating cell-free DNA and measuring changes in the level of cell-free DNA with the organ-specific methylation pattern, wherein an increase in the level of such cell-free DNA indicates organ transplant rejection.

In some embodiments, the invention includes a method of diagnosis or screening for the presence of a cancerous tumor in a patient or subject. In some embodiments, the invention includes detection of a tumor using methylation patterns of cell-free DNA using the methylation detection methods disclosed herein. In some embodiments, the invention includes detecting a tumor originating from a particular tissue or organ by detecting circulating cell-free DNA with the tissue or organ-specific methylation pattern detected using the methylation detection methods disclosed herein, wherein the presence of such cell-free DNA indicates the presence of a tumor originating from the tissue or organ. In some embodiments, the invention includes monitoring the growth or shrinkage of a tumor by periodically sampling circulating cell-free DNA and measuring changes in the level of cell-free DNA with the tumor-specific methylation pattern, wherein an increase in the level of such cell-free DNA indicates tumor growth, while a decrease in the level of such cell-free DNA indicates tumor shrinkage.

In some embodiments, the invention includes a method of monitoring the effectiveness of treatment of cancer in a patient or subject. In some embodiments, the invention includes detection of tumor dynamics correlated with treatment using methylation patterns of cell-free DNA detected using the methylation detection methods disclosed herein. In some embodiments, the invention includes detecting effects of treatment on a tumor originating from a particular tissue or organ by periodically sampling circulating cell-free DNA and measuring changes in the level of cell-free DNA with the tissue or organ-specific methylation pattern, wherein an increase in the level of such cell-free DNA indicates tumor growth and ineffectiveness of treatment, while a decrease in the level of such cell-free DNA indicates tumor shrinkage and effectiveness of treatment, and a stable level of such cell-free DNA indicates stable disease and effectiveness of treatment.

In some embodiments, the invention includes a method of diagnosis or minimal residual disease (MRD) in a cancer patient following a treatment. National Cancer Institute defines MRD as a very small number of cancer cells that remain in the body during or after treatment when the patient has no signs or symptoms of the disease. In some embodiments, the invention includes a method of detecting MRD using methylation patterns of cell-free DNA detected using the methylation detection methods disclosed herein. In some embodiments, the invention includes detecting MRD from tumor originating from a particular tissue or organ by detecting circulating cell-free DNA with the tissue or organ-specific methylation pattern, wherein the presence of such cell-free DNA indicates the presence of MRD from the tumor.

In some embodiments, the invention includes a method of diagnosis or screening for the presence or status of an autoimmune disease in a patient or subject. In some embodiments, the invention includes detection of an autoimmune disease using methylation patterns of cell-free DNA detected using the methylation detection methods disclosed herein. In some embodiments, the invention includes detecting autoimmune disease characterized by damage to a particular tissue or organ by detecting circulating cell-free DNA with the tissue or organ-specific methylation pattern, wherein the presence of such cell-free DNA indicates organ damage resulting from the autoimmune disease and the presence of the autoimmune disease. In some embodiments, the invention includes monitoring for flare-ups or remission of an autoimmune disease by periodically sampling circulating cell-free DNA and measuring changes in the level of cell-free DNA with the tissue or organ-specific methylation pattern, wherein an increase in the level of such cell-free DNA indicates increased organ damage and a flare-up of the autoimmune disease, while a decrease in the level of such cell-free DNA indicates decreased organ damage and remission of the autoimmune disease.

5-Methyl cytosine and 5-hydroxymethyl cytosine (5mC and 5hmC) are important epigenetic biomarkers with many clinical applications in oncology, prenatal testing and other fields. Until recently, base-level detection of methylation was achieved by reacting unmethylated cytosines with bisulfite followed by PCR, array hybridization or sequencing. Unmethylated cytosines (C) would read as thymine (T) after reacting with bisulfite, while methylated cytosines (5mC and 5hmC) would read as C. Unfortunately, bisulfite treatment leads to degradation of large portion of sample nucleic acid making it unsuitable for applications requiring high sensitivity. For example, the method is unsuitable for latest applications analyzing cell-free nucleic acid such as cell-free DNA.

Recently, less harsh methods for the detection of methylated cytosines have been disclosed. Rao et al. (U.S. Pat. No. 9,115,386) have discovered that the family of ten-eleven translocation dioxygenases (TET) converts 5mC into 5hmC in vitro. Liu et al. disclosed a method of stepwise oxidation of methyl-cytosines (5mC) via 5-hydroxymethyl cytosine (5hmC) to formyl cytosine (5fC) and carboxyl cytosine (5caC) using TET in the presence of Fe(II) ions and alpha-ketoglutarate. Liu, Y., et al. (2019) Bisulfite-free direct detection of 5-methylcytosine and 5-hydroxymethylcytosine at base resolution. Nat. Biotechnol. 37, 424-429.

Liu et al. further described reducing 5fC (and 5caC) using borane derivatives (such a pyridine borane, picoline borane and others) to dihydrouracil (DHU). DHU is then read by uracil-tolerant nucleic acid polymerases as T in subsequent amplification and sequencing. As a result, methylated C is read as T, while unmethylated C is unchanged. This TET and pyridine-borane based method called TAPS (TET-assisted pyridine-borane sequencing) does not cause DNA degradation as much as bisulfite treatment and allows detection of the signal directly instead of subtracting background to obtain signal. Both advantages would allow higher alignment rates, possibly lower sequencing depth and recover higher molecular diversity from the sample.

Another technique termed CAPS (Chemically Assisted Pyridine-borane Sequencing) involves the selective conversion of 5hmC to 5fC using potassium perruthenate (KRuO₄). The use of KRuO₄ as a chemical alternative to TET is known from a technique termed Oxidative Bisulfite Sequencing or oxBS-seq, see Booth M. J., et al. (2012) Quantitative sequencing of 5-methylcytosine and 5-hydroxymethylcytosine at single base resolution, Science 12 May: 934-937. The 5fC obtained by potassium perruthenate conversion becomes a favorable target for further processing by e.g. borane treatment or any other downstream method.

Yet another sequencing technique is an alternative to the reduction of 5fC with borane. This method involves forming an adduct of 5fC recognized as T. The adduct is formed with the use of malononitrile, see Zhu C., et al., (2017) Single-Cell 5-Formylcytosine Landscapes of Mammalian Early Embryos and ESCs at Single-Base Resolution, Cell Stem Cell, 20:720-731.e5.

All the above methods rely at least in part on oxidizing 5hmC and 5mC into 5fC with TET family enzymes. One other known oxidation technique involves converting 5hmC in into predominantly or exclusively 5fC with Cu(II) compound and 2,2,6,6-tetramethylpiperidine-1-oxyl (TEMPO).

Disclosed herein are methods and compositions for enzymatic oxidation of 5hmC exclusively or predominantly into 5fC. The oxidation is catalyzed with laccase, an enzyme previously known to catalyze the oxidation of phenol-containing and non-phenolic compounds under certain conditions. The inventors have discovered that surprisingly, this enzymes act in the context of nucleic acids to convert the 5-hydroxyl group of 5-hmC into a 5-formyl group.

The various aspects of the invention are described in further detail below.

In some embodiments, the invention is a method of detecting an epigenetic modification, specifically, cytosine methylation in nucleic acids. The state of the art methods of detecting methylated cytosines in nucleic acids include the following key steps: 1) oxidation of methylated cytosine; 2) conversion of the oxidized product into a form capable of being read as thymine (T) during sequencing; 3) sequencing the nucleic acids; and 4) comparing the treated and untreated sequences wherein a change from a cytosine (C) to a thymine (T) in the sequence read indicated the presence of a methylated cytosine. The instant invention comprises a new means of performing step 1) oxidizing methylated cytosine. Following the oxidation step, steps 2)-4) are performed according to the state of the art.

The present invention involves a method of manipulating nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids that may contain nucleic acids (e.g., urine, sputum, serum, blood or blood fractions, i.e., plasma, lymph, saliva, sputum, sweat, tear, cerebrospinal fluid, amniotic fluid, synovialfluid, pericardialfluid, peritonealfluid, pleuralfluid, cystic fluid, bile, gastric fluid, intestinal fluid, or fecal samples). In other embodiments, the sample is a cultured sample, e.g., a tissue culture containing cells and fluids from which nucleic acids may be isolated. In some embodiments, the nucleic acids of interest in the sample come from infectious agents such as viruses, bacteria, protozoa or fungi.

The present invention involves manipulating isolated nucleic acids isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of kits are commercially available for extracting nucleic acids (DNA or RNA) from biological samples (e.g., KAPA Express Extract (Roche Sequencing Solutions, Pleasanton, Cal.) and other similar products from BD Biosciences Clontech (Palo Alto, Cal.), Epicentre Technologies (Madison, Wisc.); Gentra Systems, (Minneapolis, Minn.); and Qiagen (Valencia, Cal.), Ambion (Austin, Tex.); BioRad Laboratories (Hercules, Cal.); and more.

In some embodiments, nucleic acids are extracted, separated by size and optionally, concentrated by epitachophoresis as described e.g., in WO2019092269 and WO2020074742.

The present invention involves detecting epigenetic modification in nucleic acids. The nucleic acid sequences that are subject to conditional epigenetic modification are the target sequences analyzed by the method disclosed herein. The same nucleic acid sequence may or may not have the epigenetic modification characterized by methylation of cytosines at the 5-position (5mC or 5hmC). In some embodiments, a set or a panel of target nucleic acids are probed for the presence of methylation. For example, as shown in Patai A V, et al. (2015) Comprehensive DNA Methylation Analysis Reveals a Common Ten-Gene Methylation Signature in Colorectal Adenomas and Carcinomas. PLOS ONE 10(8): e0133836 and in Onwuka, J. U., et al. (2020) A panel of DNA methylation signature from peripheral blood may predict colorectal cancer susceptibility. BMC Cancer 20, 692, methylation of biomarkers in a panel of methylation biomarkers is indicative of the presence of colorectal cancer in the patient. Accordingly, testing any known or future panels of methylation biomarkers for prognostic or diagnostic purposes is envisioned with the method disclosed herein.

In some embodiments, the entire genome of an organism is probed for the presence of methylation. The method of the instant invention includes detecting methylation in all sites throughout the genome of an organism to diagnose a disease or condition or predisposition to a disease or condition using the sequence analysis and artificial intelligence tools described e.g., in Shull A Y, et al., (2015) Sequencing the cancer methylome. Methods Mol Biol. 1238:627-5.

In some embodiments, it is desired to separately detect or distinguish 5mC and 5hmC in a sample. In some embodiments, it is desired to detect exclusively 5hmC in a sample by treating the sample with laccase as described herein. In other embodiments it is desired to detect only 5mC.

In one embodiment, two procedures are run in parallel on two aliquots of a sample. In one of the parallels, 5hmC is blocked while 5mC is detected exclusively by converting to a T equivalent, e.g., by TET and malononitrile procedure described in U.S. Provisional Application Ser. No. 63/147,307 filed on Feb. 9, 2021. The blocking of 5hmC takes advantage of the reactive hydroxyl group present on 5hmC but not 5mC. In some embodiments, the blocking group added to 5hmC is a sugar moiety. In some embodiments, the sugar moiety is a modified or unmodified glucose moiety and 5-glucosyl-hydroxymethyl cytosine (5ghmC) is formed. In some embodiments, addition of the blocking group is catalyzed by a glycosyltransferase, e.g., a glucosyltransferase. In some embodiments, 5hmC in nucleic acid is reacted with a modified glucose in the presence of a beta-glucosyltransferase. In some embodiments, the modified glucose is UDP-glucose and the catalyst is a bacteriophage T4 beta-glucosyltransferase (T4 BGT).

In one embodiment, two procedures are run in parallel on two aliquots of a sample. In one of the parallels, 5hmC is converted into 5fC using laccase as described herein, and 5fC is detected as T via the malononitrile process. 5mC is not reacting and is detected as C. In the second parallel, both 5hmC and 5mC are detected as T without distinction, e.g., by TET and malononitrile procedure described in in U.S. Provisional Application Ser. No. 63/147,307 filed on Feb. 9, 2021. The first of the two parallel procedures reveals 5hmC while the second of the two parallel procedures reveals 5hmC plus 5mC.

In some embodiments, the invention is a method of distinguishing 5-hydroxymethylcytosine (5hmC) from 5-methylcytosine (5mC) in nucleic acids in a sample, the method comprising: (i) separating a sample into two aliquots; (ii) in the first aliquot, contacting the nucleic acid comprising 5mC and 5hmC with a ten-eleven-translocation (TET) dioxygenase under conditions where 5hmC and 5mC are converted into 5-formyl cytosine (5fC); (iii) in the second aliquot, contacting the nucleic acid comprising 5mC and 5hmC with a laccase under conditions where 5hmC is converted into 5-formyl cytosine (5fC); (iv) contacting both aliquots separately with a moiety of formula R₁—CH₂—CN capable of reacting with 5fC in the nucleic acid to form an adduct according to the reaction scheme

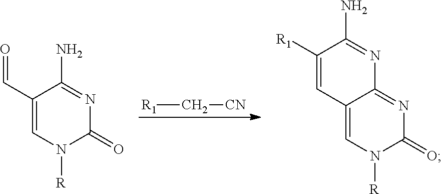

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl; (v) sequencing the nucleic acid from both aliquots separately to obtain a first and a second test sequence wherein the adduct is read as thymine (T) during sequencing; (vi) comparing the first test sequence with a reference sequence, wherein a transition from a cytosine (C) in the reference sequence to a thymine (T) in the corresponding position in the test sequence indicates the presence of 5hmC and 5mC in the nucleic acid; (vii) comparing the second test sequence with a reference sequence, wherein a transition from a cytosine (C) in the reference sequence to a thymine (T) in the corresponding position in the test sequence indicates the presence of 5hmC in the nucleic acid; and (viii) comparing the first test sequence with a second test sequence, wherein only such cytosines of the first test sequence are 5mC which are not detected as hydroxymethylated cytosines (5hmC) in the second test sequence. Alternatively, it is possible to contact both aliquots separately with a Wittig reagent.

In some embodiments, it is desired to detect 5mC and 5hmC without distinction. In this embodiment, 5mC is reacted with TET e.g., as described in U.S. Pat. No. 9,115,386 to yield 5hmC to be oxidized by laccase as described herein.

In some embodiments, the method includes a step of oxidizing methylated cytosines for downstream detection. In some embodiments, the method includes a step of converting 5-hydroxymethyl cytosine (5hmC) into 5-formyl cytosine (5fC) with laccase enzyme. In some embodiments, the oxidation takes place in the presence of a co-factor. In some embodiments, the co-factor is 2,2,6,6-tetramethylpiperidine-1-oxyl (TEMPO). In some embodiments, the oxidation takes place at low pH, e.g., pH<6. Laccase catalyzes the oxidation of phenol-containing compounds, including lignin, through the reduction of oxygen to water; the presence of mediators allows the oxidation of non-phenolic compounds like benzylic alcohols as well according to the scheme:

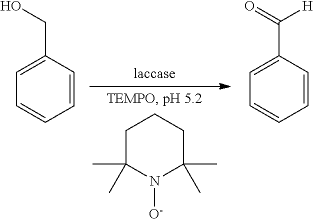

### (See Catalysis Communications 2020, 135, 105887).

In one embodiment, oxidoreductases of the laccase type (EC number 1.10.3.2) are used. In some embodiments, laccase is from a fungal source. In some embodiments, the fungal source is selected from Hexagonia tenuis, Pleurotis sajor caju, Pleutoris ostreatus, Xylaria polymorpha, Trametes hirsuta, Trametes versicolor and Coprinus spp. In some embodiments, the fungal source is selected from Hexagonia tenuis, Pleurotis sajor caju MTCC-141, Pleutoris ostreatus MTCC-1801, Xylaria polymorpha MTCC-1100, Trametes hirsuta MTCC-1171, Coprinus spp. or any other analog or equivalent thereof with similar or equivalent enzymatic activity such as alcohol dehydrogenases, alcohol oxidases, galactose oxidases, chloroperoxidases and peroxidases. In some embodiments, toluene methyl-monooxygenase (EC 1.14.15.26) and P450 monooxygenase (EC 1.14.14.1) are used to convert 5mC to 5hmC.

In some embodiments, the sample is contacted with laccase in the presence of cofactors. In some embodiments, the cofactor is 2,2,6,6-tetramethylpiperidine-1-oxyl (TEMPO). In some embodiments, the cofactors are selected from, natural cofactors for laccase selected from acetosyringone, syringaldehyde, para-coumaric acid and synthetic cofactors for laccase selected from 2,2′-azino-bis(3-ethylbenzothiazoline-6-sulfonate (ABTS), N-hydroxy type mediators like violuric acid (VLA), N-acetyl-N-phenylhydroxylamine (NHA), N-hydroxybenzotriazole (HBT), N-hydroxyphthalimide (HPI) see: Two decades of laccases: Advancing sustainability in the chemical industry: M. D. Cannatelli, A. J. Ragauskas, Chem. Rec. 2017, 17(1), 122-140.

In some embodiments, the method comprises a preliminary step of converting 5mC into 5hmC with an enzyme such as TET prior to reacting 5hmC with laccase. In some embodiments, TET and laccase are present in the same convenient “one-pot” reaction. In some embodiments, TET, laccase and malononitrile are present in the same convenient “one-pot” reaction.

In some embodiments, the invention further comprises a downstream step of detecting 5-formyl cytosine (5fC) nucleotide in a nucleic acid, wherein the 5fC is formed by the method described herein above. In some embodiments, the downstream step involves contacting a sample containing a nucleic acid comprising 5fC with an improved composition comprising a compound of formula R₁—CH₂—CN in a solvent composition, the compound being capable of reacting with 5fC in the nucleic acid to form an adduct according to the reaction scheme

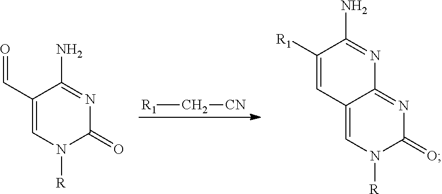

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl. The reactants of the above reaction are described e.g., in U.S. Pat. No. 10,519,184 and application Pub. No. US20200165661 by Yi et al. For example, R1 is a cyano group (CN) and the reactant is malononitrile. Alternatively, 1,3-indandione compounds can be used as the 5fC conversion reagents instead of R1-CH₂—CN (see B. Xia et al., Nature Methods 2015, 12(11), 1047-1050). Still alternatively, it is possible to react 5fC with a Wittig reagent.

In other embodiments, the downstream step involves contacting a sample containing a nucleic acid comprising 5fC with a Wittig reagent in an organic solvent, and then irradiating with ultraviolet light. The products of the reaction are detected using fluorescence recognition technology as described in WO2020155742.

In some embodiments, the compound of formula R₁—CH₂—CN is provided in a reaction mixture that enables the reaction to proceed for less than 3 hours wherein at least 90% of 5fC has formed the adduct as described in the U.S. Provisional Application Ser. No. 63/147,307 filed on Feb. 9, 2021. In some embodiments, the reaction proceeds for only 1 hour with at least 90% of 5fC forming the adduct. In some embodiments, the reaction mixture comprises an organic acid moiety. The organic acid has a formula R—COOH and R is selected from C1-C30 linear or branched alkyl, C2-C30 linear or branched alkenyl, C2-C30 linear or branched alkynyl (may comprise heteroatoms such as 0 and N), aryl or heteroaryl. In some embodiments, the reaction takes place in the presence of acetic acid. In some embodiments, the concentration of the organic acid in the reaction is between 1% and 30%, e.g., 1%, 2%, 3%, 4%, 5%, 6%, 7%, 8%, 9%, 10%, 11%, 12%, 13%, 14%, 15%, 16%, 17%, 18%, 18%, 19%, 20%, 21%, 22%, 23%, 24%, 25%, 26%, 27%, 28%, 29%, or 30%.

In some embodiments, the reaction mixture comprises a non-aqueous solvent. The non-aqueous solvent has a formula R—OH wherein R is selected from C1-C3 linear or branched alkyl and may comprise heteroatoms such as O and N. In some embodiments, the reaction takes place in methanol or ethanol. In some embodiments, the reaction takes place in 10%-100% methanol or ethanol. In some embodiments, the reaction takes place in 90% or more of methanol or ethanol.

In some embodiments, the reaction mixture comprises a compound of formula RₓNH_{y} wherein x and y are 0, 1, 2 or 3 so that x+y=3, and each R is independently selected from C1-C6 linear or branched alkyl which optionally comprise heteroatoms such as O and N, C6-C10-aryl, or 5- or 6-membered heteroaryl. In some embodiments, the compound of formula RₓNH_{y} is a primary, secondary, or tertiary amine with aliphatic or aromatic groups. In some embodiments, Rx can form with N a 5- or 6-membered cyclic heteroalkyl such as piperidine. In some embodiments, reaction takes place in the presence of triethanolamine.

Following the formation of the adduct, the nucleic acid with the adduct is subjected to sequencing. In some embodiments, sequencing is by a next-generation massively parallel sequencing process. Sequencing results in a test sequence wherein the adduct is read as thymine (T), i.e., the sequencing polymerase is able to accommodate the adduct in the strand being copied, and to incorporate an adenine (A) opposite the adduct. The method further comprises a step of comparing the test sequence with a reference sequence, wherein a change from a cytosine (C) in the reference sequence to a thymine (T) in the corresponding position in the test sequence indicates the presence of methylated and/or hydroxymethylated cytosine in the test nucleic acid.

In some embodiments, the nucleic acids in the sample are amplified prior to sequencing. In some embodiments, amplification utilizes a B-family polymerase efficiently incorporating an adenine (A) nucleotide opposite the malononitrile adduct. In this embodiment, the sequencing may proceed with any polymerase suitable for the sequencing process as the adduct has already been recognized as T by the amplification polymerase.

In some embodiments, the nucleic acid in the sample is ligated to adaptors, wherein adaptors comprise elements useful in amplification and sequencing. An adaptor comprises at least one of the following: barcode, primer binding site and ligation site.

In some embodiments, the invention is an improved method of detecting a methylated and/or hydroxymethylated cytosine nucleotide in a nucleic acid, the method comprising: (i) ligating adaptors to a nucleic acid in a sample wherein adaptors comprise amplification primer binding sites; (ii) forming a reaction mixture by contacting the sample containing adaptor-ligated nucleic acid with laccase capable of converting 5hmC in the nucleic acid into 5fC; (iii) contacting the reaction mixture with a compound of formula R₁—CH₂—CN capable of reacting with 5fC in the nucleic acid to form an adduct according to the reaction scheme

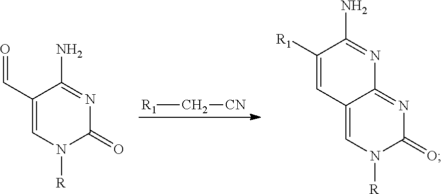

wherein R₁ is an electron-withdrawing group selected from cyano, nitro, C1-C6 alkyl carboxylic ester, unsubstituted carboxamide, C1-C6 alkyl mono-substituted and C1-C6 alkyl di-substituted carboxamide, substituted carbonyl moiety, substituted sulfonyl moiety, wherein the substitution is selected from C1-C6 linear or branched alkyl, C4-C6 cycloalkyl, phenyl, 5- or 6-membered heteroaryl and benzannulated 5- or 6-membered heteroaryl; (iv) incubating the reaction mixture for less than 3 hours wherein at least 90% of 5fC has formed the adduct; (v) amplifying the adapted nucleic acids utilizing a DNA polymerase and primers capable of binding to the primer-binding sites, wherein the DNA polymerase reads the adduct as thymine (T) during amplification; (vi) sequencing the amplified nucleic acid to obtain a test sequence; (vii) comparing the test sequence with a reference sequence, wherein a change from a cytosine (C) in the reference sequence to a thymine (T) in the corresponding position in the test sequence indicates the presence of 5mC and/or 5hmC in the nucleic acid. In some embodiments, in step (iii) the compound of formula R₁—CH₂—CN is present in a non-aqueous solvent, e.g., ethanol or methanol. In some embodiments, in step (ii) the reaction mixture is further contacted with a cofactor for laccase such as e.g., 2,2,6,6-tetramethylpiperidine-1-oxyl (TEMPO). In some embodiments, prior to step (ii), the sample is contacted with TET to convert 5mC in nucleic acids into 5hmC for reaction with laccase in step (ii). In some embodiments, in step (iii) the compound of formula R₁—CH₂—CN is present in a solution comprising an organic acid such acetic acid. In some embodiments, in step (iii) the compound of formula R₁—CH₂—CN is present in a solution comprising an amine such as triethanolamine or piperidine. Alternatively, it is possible to use a Wittig reagent in step (iii).

In some embodiments, the invention includes a step of amplifying nucleic acids. In some embodiments, amplification occurs prior to the sequencing step. In some embodiments, amplification occurs after the step of forming an adduct of 5fC and malononitrile. In some embodiments, amplification occurs after the step of reduction of oxidized methylated cytosine with a borane derivative. In some embodiments, amplification occurs prior to the target enrichment step. The amplification utilizes an upstream primer and a downstream primer. In some embodiments, both primers are target specific primers, i.e., primers comprising a sequence complementary to the target sequence of the methylation biomarker. In other embodiments, one or both primers are universal primers. In some embodiments, universal primer binding sites are present in adaptors ligated to the target sequenced as described herein. In some embodiments, an universal primer binding site is present in the 5′-region (tail) of a target-specific primer. Accordingly, after one or more rounds of primer extension with a tailed target-specific primer, an universal primer may be used for subsequent rounds of amplification. In some embodiments, an universal primer in paired with another universal primer (of the same or different sequence). In other embodiments, an universal primer is paired with a target-specific primer.

In some embodiments, the invention involves a nucleic acid polymerase. Nucleic acid polymerases used in amplification and sequencing are known and commercially available from multiple sources. In some embodiments, the instant invention involves copying a strand comprising a 5fC adduct formed as described herein. Such copying requires a polymerase accommodating the 5fC adduct. In some embodiments, the polymerase is a B-family polymerase. In some embodiments, the polymerases is able to copy a strand comprising a 5fC adduct by recognizing the adduct as T (i.e., incorporating an A opposite the adduct). Polymerases able to accommodate the 5fC adduct described herein include DNA polymerases known to accommodate uracil (U) in a DNA strand. The polymerase may be a naturally-occurring or an engineered polymerase. In some embodiments, the polymerase is isolated from hyperthermophilic archaea e.g., genus Pyrococcus (e.g., Pyrococcus furious) or genus Thermus (e.g., Thermus aquaticus). In some embodiments, the polymerase is isolated from mesophilic archaea, e.g., genus Metanosarcina (e.g., Methanosarcina acetivorans). Examples of engineered uracil-tolerant polymerases include KAPA HiFi Uracil+ DNA polymerase (Roche Sequencing Solutions, Pleasanton, Cal.), Takara Terra (Takara Bio USA, Mountain View, Cal.), and EpiMark© Hot Start Taq DNA polymerase (New England Biolabs, Waltham, Mass.).

In some embodiments, the DNA polymerase is a type A DNA polymerase (DNA-dependent DNA polymerase). Some DNA polymerases possess limited terminal transferase activity (Taq polymerase adding a single dA at the 3′-end of the copy strand). Other DNA polymerases do not possess detectable terminal transferase activity. In such embodiments, a separate terminal transferase enzyme is used to add non-templated nucleotides to the 3′-end of the copy strand.

In some embodiments, the DNA polymerase is a Hot Start polymerase or a similar conditionally activated polymerase. For the amplification step, a thermostable DNA polymerase is used, for example the polymerase is a Taq or Taq-derived polymerase (e.g., KAPA 2G polymerase from KAPA Biosystems, Wilmington, Mass.).

In some embodiments, the invention utilizes an adaptor added to one or both ends of a nucleic acid or nucleic acid strand. Adaptors of various shapes and functions are known in the art (see e.g., PCT/EP2019/05515 filed on Feb. 28, 2019, U.S. Pat. Nos. 8,822,150 and 8,455,193). In some embodiments, the function of an adaptor is to introduce desired elements into a nucleic acid. The adaptor-borne elements include at least one of nucleic acid barcode, primer binding site or a ligation-enabling site.

The adaptor may be double-stranded, partially single stranded or single stranded. In some embodiments, a Y-shaped, a hairpin adaptor or a stem-loop adaptor is used wherein the double-stranded portion of the adaptor is ligated to the double stranded nucleic acid formed as described herein.

In some embodiments, the adaptor molecules are in vitro synthesized artificial sequences. In other embodiments, the adaptor molecules are in vitro synthesized naturally-occurring sequences. In yet other embodiments, the adaptor molecules are isolated naturally occurring molecules or isolated non naturally-occurring molecules.

The double-stranded or partially double-stranded adaptor oligonucleotide can have overhangs or blunt ends. In some embodiments, the double-stranded DNA may comprise blunt ends to which a blunt-end ligation can be applied to ligate a blunt-ended adaptor. In other embodiments, the blunt ended DNA undergoes A-tailing where a single A nucleotide is added to the blunt ends to match an adaptor designed to have a single T nucleotide extending from the blunt end to facilitate ligation between the DNA and the adaptor. Commercially available kits for performing adaptor ligation include AVENIO ctDNA Library Prep Kit or KAPA HyperPrep and HyperPlus kits (Roche Sequencing Solutions, Pleasanton, CA). In some embodiments, the adaptor ligated (adapted) DNA may be separated from excess adaptors and unligated DNA.

In some embodiments, the invention includes the use of a barcode. In some embodiments, the method of detecting epigenetic modifications includes sequencing. The nucleic acid processed as described herein is subjected to sequencing; preferably, massively parallel single molecule sequencing. Analyzing individual molecules by massively parallel sequencing typically requires a separate level of barcoding for sample identification and error correction. The use of molecular barcodes such as described in U.S. Pat. Nos. 7,393,665, 8,168,385, 8,481,292, 8,685,678, and 8,722,368. A unique molecular barcode is added to each molecule to be sequenced to mark molecule and its progeny (e.g., the original molecule and its amplicons generated by PCR). The unique molecular barcode (UTD) has multiple uses including counting the number of original target molecules in the sample and error correction (Newman, A., et al., (2014) An ultrasensitive method for quantitating circulating tumor DNA with broad patient coverage, Nature Medicine doi:10.1038/nm.3519).

In some embodiments, unique molecular barcodes (UIDs) are used for sequencing error correction. The entire progeny of a single target molecule is marked with the same barcode and forms a barcoded family. A variation in the sequence not shared by all members of the barcoded family is discarded as an artefact. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments of the invention, the adaptor ligated to one or both ends of the barcoded target nucleic acid comprises one or more barcodes used in sequencing. A barcode can be a UID or a multiplex sample ID (MID or SID) used to identify the source of the sample where samples are mixed (multiplexed). The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. In some embodiments, the number of UIDs in the reaction can be in excess of the number of molecules to be labelled. A person of ordinary skill would recognize that the number of barcodes depends on the complexity of the sample (i.e., expected number of unique target molecules) and would be able to create a suitable number of barcodes for each experiment.

In some embodiments, the method involves forming a library comprising nucleic acids from a sample. The library consists of a plurality of nucleic acids ready for sequencing or another type of detection method, e.g., PCR. A library can be stored and used multiple times for further processing such as amplification or sequencing of the nucleic acids in the library. In some embodiments, the library is the input nucleic acid in which methylation is detected by the method described herein. In other embodiments, the library is formed from nucleic acids that have undergone the methylation detection reactions described herein.

In some embodiments, the nucleic acids processed for detection of epigenetic modifications according to the method described herein are sequenced. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (U.S. Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeq™, MiSeq™, or Genome Analyzer, each available from Ilumina), sequencing by ligation (e.g., SOLiD™, Life Technologies), ion semiconductor sequencing (e.g., Ion Torrent™, Life Technologies), and SMRT® sequencing (e.g., Pacific Biosciences).

Commercially available sequencing technologies include: sequencing-by-hybridization platforms from Affymetrix Inc. (Sunnyvale, Calif.), sequencing-by-synthesis platforms from Ilumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology from Roche Sequencing Solutions, Santa Clara, Cal.), and Oxford Nanopore Technologies (Oxford, UK).

In some embodiments, the sequencing step involves sequence aligning. In some embodiments, aligning is used to determine a consensus sequence from a plurality of sequences, e.g., a plurality having the same unique molecular ID (UID). The molecular ID is a barcode that can be added to each molecule prior to sequencing or if amplification step is included, prior to the amplification step. In some embodiments, a UID is present in the 5′-portion of the RT primer. Similarly, a UID can be present in the 5′-end of the last barcode subunit to be added to the compound barcode. In other embodiments, a UID is present in an adaptor and is added to one or both ends of the target nucleic acid by ligation.

In some embodiments, a consensus sequence is determined from a plurality of sequences all having an identical UID. The sequences having an identical UID are presumed to derive from the same original molecule through amplification. In other embodiments, UID is used to eliminate artifacts, i.e., variations existing in the progeny of a single molecule (characterized by a particular UID). Such artifacts resulting from PCR errors or sequencing errors can be eliminated using UIDs.

In some embodiments, the number of each sequence in the sample can be quantified by quantifying relative numbers of sequences with each UID among the population having the same multiplex sample ID (MID). Each UID represents a single molecule in the original sample and counting different UIDs associated with each sequence variant can determine the fraction of each sequence variant in the original sample, where all molecules share the same MID. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the invention is a kit including components and tools for performing an improved method of detecting DNA methylation described herein. In some embodiments, the kit includes reagents for detecting cytosine methylation in nucleic acids by performing in vitro oxidation of 5-hydroxymethyl cytosine (5hmC) to 5-formyl cytosine (5fC). In some embodiments, the kit further comprises reagents for detecting 5-formyl cytosine (5fC) in nucleic acids.

In some embodiments, the kit comprises a laccase enzyme. In some embodiments, laccase is from a fungal source. In some embodiments, the fungal source is selected from Hexagonia tenuis, Pleurotis sajor caju, Pleutoris ostreatus, Xylaria polymorpha, Trametes hirsuta, Trametes versicolor and Coprinus spp. In some embodiments, the fungal source is selected from Hexagonia tenuis, Pleurotis sajor caju MTCC-141, Pleutoris ostreatus MTCC-1801, Xylaria polymorpha MTCC-1100, Trametes hirsuta MTCC-1171, Coprinus spp. or any other analog or equivalent thereof with similar or equivalent enzymatic activity such as F.

In some embodiments, the kit further includes a co-factor for laccase oxidation. In some embodiments, the cofactor is selected from 2,2,6,6-tetramethylpiperidine-1-oxyl (TEMPO), acetosyringone, syringaldehyde, para-coumaric acid 2,2′-azino-bis(3-ethylbenzothiazoline-6-sulfonate (ABTS), violuric acid (VLA), N-acetyl-N-phenylhydroxylamine (NHA), N-hydroxybenzotriazole (HBT), and N-hydroxyphthalimide (HPI).

In some embodiments, the kit further includes ten-eleven translocation dioxygenase (TET). In some embodiments, TET is selected from mouse TET1, TET2 or TET3 (mTET1, 2 or 3), human TET1, TET2 or TET3 (hTET1, 2 or 3), Naegleria TET (NgTET), Coprinopsis cinerea (CcTET) or any other analog or equivalent thereof with similar or equivalent enzymatic activity.

In some embodiments, the kit further includes malononitrile. In some embodiments, malononitrile is present in a non-aqueous solvent. The non-aqueous solvent is selected from ethanol and methanol. In other embodiments, instead of including the non-aqueous solvent, the kit includes instructions on using the non-aqueous solvent (such as ethanol or methanol) in a method of detecting DNA methylation with malononitrile as described herein. In some embodiments, the kit further comprises an organic acid and a primary, a secondary or a tertiary amine. The organic acid may be acetic acid and the amine may be triethanolamine. In other embodiments, the kit includes instructions on using the organic acid and the amine (such as acetic acid and triethanolamine or piperidine) in a method of detecting DNA methylation with malononitrile as described herein. In some embodiments, the kit further includes a buffer such as MES or TRIS.

In some embodiments, the kit further includes reagents for distinguishing 5mC in nucleic acids from 5hmC by protecting 5hmC while 5mC is chemically reacted. In some embodiments, the kit includes a glucose compound and a glucosyltransferase capable of transferring the glucose moiety to the 5-hydroxyl moiety of 5hmC to form 5-glucosylhydroxymethyl cytosine (5ghmC). In some embodiments, the kit includes a beta-glucosyltransferase (BGT) and a UDP-glucose. In some embodiments, the BGT is T4 BGT.

In some embodiments, the method further comprises assessment of a status of a subject (e.g., a patient) based on the methylation status of one or more genetic loci in the patient's genome. In some embodiments, the method comprises determining in the patient's sample, the genomic location and optionally, amount of methylated cytosines (5mC and/or 5hmC) in the genome. In some embodiments, genetic loci known to be biomarkers of disease are assessed for methylation. The method further comprises diagnosis of disease or condition in the patient or selecting or changing a treatment based on the presence or amount of methylation in the nucleic acid isolated from the patient.

Several methods exists for identifying disease or condition-specific methylation loci that can be assessed for methylation using the methods disclosed herein, see e.g., US20200385813 “Systems and methods for estimating cell source fractions using methylation information;” US20200239965 “Source of origin deconvolution based on methylation fragments in cell-free DNA samples;” US20190287652 “Anomalous fragment detection and classification” (methylation markers indicating disease state); US20190316209 “Multi-assay prediction model for cancer detection;” US20190390257A1 “Tissue-specific methylation marker;” WO2011/070441 “Categorization of DNA samples;” WO2011/101728 “Identification of source of DNA samples;” WO2020/188561 “Methods and systems for detecting methylation changes in DNA samples.”

In some embodiments, the invention includes a method of detecting tissue-specific DNA methylation patterns using the methylation detection methods disclosed herein. In one aspect of this embodiment, the method may further include identifying a tissue of origin of the methylated DNA present in the sample. In some embodiments, the method further includes identifying a tissue of origin of cell-free DNA isolated from blood. In another aspect of this embodiment, the invention includes detection of organ failure or organ injury, including organ transplant rejection in a transplant recipient using methylation patterns of cell-free DNA. The invention includes detecting circulating cell-free DNA with the organ-specific methylation pattern, wherein the presence of such cell-free DNA indicates organ transplant rejection. In some embodiments, the invention includes monitoring for transplant rejection by periodically sampling circulating cell-free DNA and measuring changes in the level of cell-free DNA with the organ-specific methylation pattern, wherein an increase in the level of such cell-free DNA indicates organ transplant rejection.

In some embodiments, the invention includes a method of diagnosis or screening for the presence of a cancerous tumor in a patient or subject. In some embodiments, the invention includes detection of a tumor using methylation patterns of cell-free DNA using the methylation detection methods disclosed herein. In some embodiments, the invention includes detecting a tumor originating from a particular tissue or organ by detecting circulating cell-free DNA with the tissue or organ-specific methylation pattern detected using the methylation detection methods disclosed herein, wherein the presence of such cell-free DNA indicates the presence of a tumor originating from the tissue or organ. In some embodiments, the invention includes monitoring the growth or shrinkage of a tumor by periodically sampling circulating cell-free DNA and measuring changes in the level of cell-free DNA with the tumor-specific methylation pattern, wherein an increase in the level of such cell-free DNA indicates tumor growth, while a decrease in the level of such cell-free DNA indicates tumor shrinkage.

In some embodiments, the invention includes a method of monitoring the effectiveness of treatment of cancer in a patient or subject. In some embodiments, the invention includes detection of tumor dynamics correlated with treatment using methylation patterns of cell-free DNA detected using the methylation detection methods disclosed herein. In some embodiments, the invention includes detecting effects of treatment on a tumor originating from a particular tissue or organ by periodically sampling circulating cell-free DNA and measuring changes in the level of cell-free DNA with the tissue or organ-specific methylation pattern, wherein an increase in the level of such cell-free DNA indicates tumor growth and ineffectiveness of treatment, while a decrease in the level of such cell-free DNA indicates tumor shrinkage and effectiveness of treatment, and a stable level of such cell-free DNA indicates stable disease and effectiveness of treatment.

In some embodiments, the invention includes a method of diagnosis or minimal residual disease (MRD) in a cancer patient following a treatment. National Cancer Institute defines MRD as a very small number of cancer cells that remain in the body during or after treatment when the patient has no signs or symptoms of the disease. In some embodiments, the invention includes a method of detecting MRD using methylation patterns of cell-free DNA detected using the methylation detection methods disclosed herein. In some embodiments, the invention includes detecting MRD from tumor originating from a particular tissue or organ by detecting circulating cell-free DNA with the tissue or organ-specific methylation pattern, wherein the presence of such cell-free DNA indicates the presence of MRD from the tumor.

In some embodiments, the invention includes a method of diagnosis or screening for the presence or status of an autoimmune disease in a patient or subject. In some embodiments, the invention includes detection of an autoimmune disease using methylation patterns of cell-free DNA detected using the methylation detection methods disclosed herein. In some embodiments, the invention includes detecting autoimmune disease characterized by damage to a particular tissue or organ by detecting circulating cell-free DNA with the tissue or organ-specific methylation pattern, wherein the presence of such cell-free DNA indicates organ damage resulting from the autoimmune disease and the presence of the autoimmune disease. In some embodiments, the invention includes monitoring for flare-ups or remission of an autoimmune disease by periodically sampling circulating cell-free DNA and measuring changes in the level of cell-free DNA with the tissue or organ-specific methylation pattern, wherein an increase in the level of such cell-free DNA indicates increased organ damage and a flare-up of the autoimmune disease, while a decrease in the level of such cell-free DNA indicates decreased organ damage and remission of the autoimmune disease.

### REFERENCES

1. Keciek, A., Catalysis Communications 2020, 135, 105887 Evaluation of alcohols as substrates for the synthesis of 3,4-dihydropyrimidin-2(1H)-ones under environmentally friendly conditions
2. Safaei, E. et al., Polyhydron 2016, 106, 153-162 TEMPO-mediated aerobic oxidation of alcohols using copper(II) complex of bis(phenol) di-amine ligand as biomimetic model for Galactose oxidase enzyme
3. Sattler, J. H.; Kroutil, W., edited by Whittall, J. et al.; Practical Methods for Biocatalysis and Biotransformations 2 (2012), 177-179 Chemoselective oxidation of primary alcohols to aldehydes
4. Wu, J. et al., Current Microbiology 2011, 62(4), 1123-1127 Highly selective oxidation of Benzyl alcohol using engineered Gluconobacter oxydans in biphasic system
5. Jain, A. N. et al., Biotechnology Letters 2010, 32(11), 1649-1654 Bioproduction of benzaldehyde in a solid-liquid 2-phase partitioning bioreactor using Pichia pastoris
6. Villa, R. et al, Tetrahedron Letters 2002, 43(34), 6059-6061 Chemoselective oxidation of primary alcohols to aldehydes with Gluconobacter oxydans
7. Buhler, B. et al., Applied and Environmental Microbiology 2002, 68(2), 560-568 Characterization and application of xylene monooxygenase for multistep biocatalysis
8. Fabbrini M. et al., Journal of Molecular Catalysis B: Enzymatic 2002, 16(5-6), 231-240 Comparing the catalytic efficiency of some mediators of laccase
9. Samra, B. K. et al., Biocatalysis and Biotransformation 1999, 17(5), 381-391 Chloroperoxidase catalyzed oxidation of benzyl alcohol using tert-butyl hydroperoxide oxidant in organic media
10. McSkimming, A. et al., Journal of the American Chemical Society 2018, 140(4), 1223-1226 Functional synthetic model for the lanthanide-dependent quinoid alcohol dehydrogenase active site
11. Safaei, Elham et al., Journal of Molecular Structure 2017, 1133, 526-533 Copper(II) complex of new non-innocent Oaminophenol-based ligand as biomimetic model for galactose oxidase enzyme in aerobic oxidation of alcohols
12. Baciocchi, Enrico et al., Chemical Communications (Cambridge) 1999, (17), 1715-1716 Prochiral selectivity and deuterium kinetic isotope effect in the oxidation of benzyl alcohol catalyzed by chloroperoxidase
13. Orbegozo, Thomas et al., Tetrahedron 2009, 65(34), 6805-6809 Biocatalytic oxidation of benzyl alcohol to benzaldehyde via hydrogen transfer
14. Duff, Sheldon J. B. and Murray, William D., U.S., 5010005, 23 Apr. 1991 Enzymic oxidation of higher alcohols in two phase systems with alcohol oxidase
15. Duff, Sheldon J. B. and Murray, William D., Biotechnology and Bioengineering 1989, 34(2), 153-9 Oxidation of benzyl alcohol by whole cells of Pichia pastoris and by alcohol oxidase in aqueous and nonaqueous reaction media
16. Fritz-Langhals, E.; Kunath, B., Tetrahedron Letters 1998, 39(33), 5955-5956 Synthesis of aromatic aldehydes by laccase-mediator assisted oxidation
17. Gandolfi, R. et al., Tetrahedron Letters 2001, 42(3), 513-514 An easy and efficient method for the production of carboxylic acids and aldehydes by microbial oxidation of primary alcohols
18. Geigert, John et al., Biochemical and Biophysical Research Communications 1983, 114(3), 1104-8 Peroxide oxidation of primary alcohols to aldehydes by chloroperoxidase catalysis
19. Chaurasia, P. K. et al., International Journal of Research in Chemistry and Environment 2013, 3(1), 188-197 Selective biotransformation of aromatic methyl groups to aldehyde groups using crude laccase of Pleurotus ostreatus MTCC-1803
20. Chaurasia, P. K. et al., BioChemistry: An Indian Journal 2012, 6(7), 237-242 Application of crude laccase of Xylaria polymorpha MTCC-1100 in selective oxidation of aromatic methyl group to aldehyde group
21. Park, Jin-Byung; Clark, Douglas S., Biotechnology and Bioengineering 2006, 94(1), 189-192 New reaction system for hydrocarbon oxidation by chloroperoxidase
22. Nueske, J. et al., DE 102004047774 A1 20060330 Process for the enzymatic hydroxylation of non-activated hydrocarbons
23. Hauer, B. et al., WO 2003031634 A1 20030417 Method for selective oxidation of substituted toluenes by Coprinus peroxidases
24. Maruyama, T. et al., Journal of Molecular Catalysis B: Enzymatic 2003, 21(4-6), 211-219 Oxidation of both termini of p- and m-xylene by Escherichia coli transformed with xylene monooxygenase gene
25. Buhler, B. et al., Applied and Environmental Microbiology 2002, 68(2), 560-568 Characterization and application of xylene monooxygenase for multistep biocatalysis
26. Russ, R. et al., Tetrahedron Letters 2002, 43(5), 791-793 Benzylic bio-oxidation of various toluenes to aldehydes by peroxidase.
27. Potthast, A. et al., Journal of Organic Chemistry 1995, 60(14), 4320-1 Selective Enzymic Oxidation of Aromatic Methyl Groups to Aldehydes

Some embodiments of the disclosure are directed to a method for detecting 5-hydroxymethylcytosine (5hmC) in a target nucleic acid from a sample, wherein the method comprises the following steps: (a) contacting the target nucleic acid with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC); (b) contacting the nucleic acid comprising one or more 5fC of step (a) with malononitrile, wherein the malononitrile converts 5fC to 5fC-M adduct, thereby producing a nucleic acid comprising one or more 5fC-M adduct; (c) contacting the nucleic acid comprising one or more 5fC-M adduct of step (b) with a polymerase, wherein the polymerase converts 5fC-M adduct to Thymine (T), thereby producing a nucleic acid comprising one or more T; and (d) sequencing the nucleic acid comprising one or more T of step (c), wherein if a T is detected at a position in the nucleic acid comprising one or more T of step (c) where a 5hmC was originally present in the target nucleic acid, then 5hmC has been detected in the target nucleic acid. In a related embodiment, the target nucleic acid is contacted with laccase in step (a), and wherein step (a) occurs in less than 22 hours, or step (a) occurs in less than 5 hours, or step (a) occurs in less than 4 hours, or step (a) occurs in less than 3 hours, or step (a) occurs in in 3 hours. In a related embodiment, step (a) occurs at around 25° C., or step (a) occurs at 25° C., or step (a) occurs at around 37° C., or step (a) occurs at 37° C. In another embodiment, the target nucleic acid is contacted with Cu(II)/TEMPO in step (a), and wherein step (a) occurs in less than 24 hours, or step (a) occurs in 22 hours. In another embodiment, step (b) occurs at around 60° C. In a related embodiment, step (b) occurs at 60° C., and/or step (b) occurs in a reaction mixture, wherein the reaction mixture comprises a buffer. In one embodiment, the buffer comprises 25 mM Tris. In another embodiment, the buffer is at a pH of around 8. In another embodiment, the nucleic acid comprising one or more 5fC is contacted with malononitrile for 1.5 hours. In another embodiment, the method further comprises an additional step between step (a) and step (b), wherein the additional step between step (a) and step (b) comprises contacting the nucleic acid comprising one or more 5fC with NaOH. In a related embodiment, the nucleic acid comprising one or more 5fC is contacted with malononitrile in step (b) for less than 1 hour. In another embodiment, the nucleic acid comprising one or more 5fC is contacted with malononitrile in step (b) for about 1 hour. In another embodiment, the nucleic acid comprising one or more 5fC is contacted with malononitrile in step (b) for less than 30 minutes. In another embodiment, the nucleic acid comprising one or more 5fC is contacted with malononitrile in step (b) for about 30 minutes. In another embodiment, step (c) occurs in a reaction mixture, wherein the reaction mixture comprises a buffer. In another embodiment, the buffer comprises the following components: (i) 5% dimethyl sulfoxide (DMSO); (ii) 0.85M Betaine; (iii) 70 mM tetramethylammonium chloride (TMAC); (iv) 2.1 mM dATP; (v) 2.25 mM MgCl2; and (vi) 15 mM ammonium sulfate.

Another embodiment of the disclosure is directed to a method for detecting 5-methylcytosine (5mC) in a target nucleic acid from a sample, wherein the method comprises the following steps: (a) contacting the target nucleic acid with ten-eleven-translocation (TET), wherein the TET converts 5mC to 5-hydroxymethylcytosine (5hmC), thereby producing a nucleic acid comprising one or more 5hmC; (b) contacting the nucleic acid comprising one or more 5hmC of step (a) with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC); (c) contacting the nucleic acid comprising one or more 5fC of step (b) with malononitrile, wherein the malononitrile converts 5fC to 5fC-malononitrile adduct (55fC-M adduct), thereby producing a nucleic acid comprising one or more 5fC-M adduct; (d) contacting the nucleic acid comprising one or more 5fC-M adduct of step (c) with a polymerase, wherein the polymerase converts 5fC-M adduct to Thymine (T), thereby producing a nucleic acid comprising one or more T; and (e) sequencing nucleic acid comprising one or more T of step (d), wherein if a T is detected at a position in the nucleic acid comprising one or more T of step (d) where a 5hmC was originally present in the target nucleic acid, then 5hmC has been detected in the target nucleic acid. In another embodiment, step (a) occurs in a reaction mixture, wherein the reaction mixture comprises a buffer comprising an amine catalyst. In another embodiment, the amine catalyst is 2-amino-5-methoxybenzoic acid. In another embodiment, the buffer comprises sodium phosphate and has a pH of around 5.2. In another embodiment, the amine catalyst is 2-(aminomethyl)imidazole dihydrochloride. In another embodiment, the buffer comprises Tris and has a pH of around 8. In another embodiment, the target nucleic acid is contacted with laccase in step (b). In another embodiment, step (b) occurs in less than 22 hours. In another embodiment, step (b) occurs in less than 5 hours. In another embodiment, step (b) occurs in less than 4 hours. In another embodiment, step (b) occurs in less than 3 hours. In another embodiment, step (b) occurs in 3 hours. In another embodiment, step (b) occurs at around 25° C. In another embodiment, step (b) occurs at 25° C. In another embodiment, step (b) occurs at around 37° C. In another embodiment, step (b) occurs at 37° C. In another embodiment, step (a) and step (b) are combined in a single step. In another embodiment, the combination of step (a) and step (b) occurs in a reaction mixture, wherein the reaction mixture comprises a buffer, and wherein the buffer comprises both TET and laccase. In another embodiment, the target nucleic acid is contacted with TET, wherein the TET converts 5mC to 5hmC, thereby producing a nucleic acid comprising one or more 5hmC; and wherein the laccase converts 5hmC to 5fC, thereby producing a nucleic acid comprising one or more 5fC. In another embodiment, the target nucleic acid is contacted with Cu(II)/TEMPO in step (b). In another embodiment, step (b) occurs in less than 24 hours. In another embodiment, step (b) occurs in 22 hours. In another embodiment, step (c) occurs at around 60° C. In another embodiment, step (c) occurs at 60° C. In another embodiment, step (c) occurs in a reaction mixture, wherein the reaction mixture comprises a buffer. In another embodiment, the buffer comprises 25 mM Tris. In another embodiment, the buffer is at a pH of around 8. In another embodiment, the nucleic acid comprising one or more 5fC is contacted with malononitrile for 1.5 hours. In another embodiment, the method further comprises an additional step between step (b) and step (c), wherein the additional step between step (b) and step (c) comprises contacting the nucleic acid comprising one or more 5fC with NaOH. In another embodiment, the nucleic acid comprising one or more 5fC is contacted with malononitrile in step (c) for less than 1 hour. In another embodiment, the nucleic acid comprising one or more 5fC is contacted with malononitrile in step (c) for about 1 hour. In another embodiment, the nucleic acid comprising one or more 5fC is contacted with malononitrile in step (c) for less than 30 minutes. In another embodiment, the nucleic acid comprising one or more 5fC is contacted with malononitrile in step (c) for about 30 minutes. In another embodiment, step (d) occurs in a reaction mixture, wherein the reaction mixture comprises a buffer. In another embodiment, the buffer comprises the following components: (i) 5% dimethyl sulfoxide (DMSO); (ii) 0.85M Betaine; (iii) 70 mM tetramethylammonium chloride (TMAC); (iv) 2.1 mM dATP; (v) 2.25 mM MgCl2; and (vi) 15 mM ammonium sulfate.

Another embodiment of the disclosure is directed to a method for converting 5-hydroxymethylcytosine (5hmC), in a target nucleic acid from a sample, to Thymine (T), wherein the method comprises the following steps: (a) contacting the target nucleic acid with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC); (b) contacting the nucleic acid comprising one or more 5fC of step (a) with malononitrile, wherein the malononitrile converts 5fC to 5fC-M adduct, thereby producing a nucleic acid comprising one or more 5fC-M adduct; and (c) contacting the nucleic acid comprising one or more 5fC-M adduct of step (b) with a polymerase, wherein the polymerase converts 5fC-M adduct to Thymine (T), thereby producing a nucleic acid comprising one or more T; and wherein the 5hmC in a target nucleic acid has been converted to T. In another embodiment, the target nucleic acid is contacted with laccase in step (a). In another embodiment, step (a) occurs in less than 22 hours. In another embodiment, step (a) occurs in less than 5 hours. In another embodiment, step (a) occurs in less than 4 hours. In another embodiment, step (a) occurs in less than 3 hours. In another embodiment, step (a) occurs in 3 hours. In another embodiment, step (a) occurs at around 25° C. In another embodiment, step (a) occurs at 25° C. In another embodiment, step (a) occurs at around 37° C. In another embodiment, step (a) occurs at 37° C. In another embodiment, the target nucleic acid is contacted with Cu(II)/TEMPO in step (a). In another embodiment, step (a) occurs in less than 24 hours. In another embodiment, step (a) occurs in 22 hours. In another embodiment, step (b) occurs at around 60° C. In another embodiment, step (b) occurs at 60° C. In another embodiment, step (b) occurs in a reaction mixture, wherein the reaction mixture comprises a buffer. In another embodiment, the buffer comprises 25 mM Tris. In another embodiment, the buffer is at a pH of around 8. In another embodiment, the nucleic acid comprising one or more 5fC is contacted with malononitrile for 1.5 hours. In another embodiment, the method further comprises an additional step between step (a) and step (b), wherein the additional step between step (a) and step (b) comprises contacting the nucleic acid comprising one or more 5fC with NaOH. In another embodiment, the nucleic acid comprising one or more 5fC is contacted with malononitrile in step (b) for less than 1 hour. In another embodiment, the nucleic acid comprising one or more 5fC is contacted with malononitrile in step (b) for about 1 hour. In another embodiment, the nucleic acid comprising one or more 5fC is contacted with malononitrile in step (b) for less than 30 minutes. In another embodiment, the nucleic acid comprising one or more 5fC is contacted with malononitrile in step (b) for about 30 minutes. In another embodiment, step (c) occurs in a reaction mixture, wherein the reaction mixture comprises a buffer. In another embodiment, the buffer comprises the following components: (i) 5% dimethyl sulfoxide (DMSO); (ii) 0.85M Betaine; (iii) 70 mM tetramethylammonium chloride (TMAC); (iv) 2.1 mM dATP; (v) 2.25 mM MgCl2; and (vi) 15 mM ammonium sulfate.

Another embodiment of the disclosure is directed to a method for converting 5-methylcytosine (5mC), in a target nucleic acid from a sample, to Thymine (T), wherein the method comprises the following steps: (a) contacting the target nucleic acid with ten-eleven-translocation (TET), wherein the TET converts 5mC to 5-hydroxymethylcytosine (5hmC), thereby producing a nucleic acid comprising one or more 5hmC; (b) contacting the nucleic acid comprising one or more 5hmC of step (a) with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC); (c) contacting the nucleic acid comprising one or more 5fC of step (b) with malononitrile, wherein the malononitrile converts 5fC to 5fC-malononitrile adduct (5fC-M adduct), thereby producing a nucleic acid comprising one or more 5fC-M adduct; (d) contacting the nucleic acid comprising one or more 5fC-M adduct of step (c) with a polymerase, wherein the polymerase converts 5fC-M adduct to Thymine (T), thereby producing a nucleic acid comprising one or more T; and wherein the 5mC in a target nucleic acid has been converted to T. In another embodiment, step (a) occurs in a reaction mixture, wherein the reaction mixture comprises a buffer comprising an amine catalyst. In another embodiment, the amine catalyst is 2-amino-5-methoxybenzoic acid. In another embodiment, the buffer comprises sodium phosphate and has a pH of around 5.2. In another embodiment, the amine catalyst is 2-(aminomethyl)imidazole dihydrochloride. In another embodiment, the buffer comprises Tris and has a pH of around 8. In another embodiment, the target nucleic acid is contacted with laccase in step (b). In another embodiment, step (b) occurs in less than 22 hours. In another embodiment, step (b) occurs in less than 5 hours. In another embodiment, step (b) occurs in less than 4 hours. In another embodiment, step (b) occurs in less than 3 hours. In another embodiment, step (b) occurs in 3 hours. In another embodiment, step (b) occurs at around 25° C. In another embodiment, step (b) occurs at 25° C. In another embodiment, step (b) occurs at around 37° C. In another embodiment, step (b) occurs at 37° C. In another embodiment, step (a) and step (b) are combined in a single step. In another embodiment, the combination of step (a) and step (b) occurs in a reaction mixture, wherein the reaction mixture comprises a buffer, and wherein the buffer comprises both TET and laccase. In another embodiment, the target nucleic acid is contacted with TET, wherein the TET converts 5mC to 5hmC, thereby producing a nucleic acid comprising one or more 5hmC; and wherein the laccase converts 5hmC to 5fC, thereby producing a nucleic acid comprising one or more 5fC. In another embodiment, the target nucleic acid is contacted with Cu(II)/TEMPO in step (b). In another embodiment, step (b) occurs in less than 24 hours. In another embodiment, step (b) occurs in 22 hours. In another embodiment, step (c) occurs at around 60° C. In another embodiment, step (c) occurs at 60° C. In another embodiment, step (c) occurs in a reaction mixture, wherein the reaction mixture comprises a buffer. In another embodiment, the buffer comprises 25 mM Tris. In another embodiment, the buffer is at a pH of around 8. In another embodiment, the nucleic acid comprising one or more 5fC is contacted with malononitrile for 1.5 hours. In another embodiment, the method further comprises an additional step between step (b) and step (c), wherein the additional step between step (b) and step (c) comprises contacting the nucleic acid comprising one or more 5fC with NaOH. In another embodiment, the nucleic acid comprising one or more 5fC is contacted with malononitrile in step (c) for less than 1 hour. In another embodiment, the nucleic acid comprising one or more 5fC is contacted with malononitrile in step (c) for about 1 hour. In another embodiment, the nucleic acid comprising one or more 5fC is contacted with malononitrile in step (c) for less than 30 minutes. In another embodiment, the nucleic acid comprising one or more 5fC is contacted with malononitrile in step (c) for about 30 minutes. In another embodiment, step (d) occurs in a reaction mixture, wherein the reaction mixture comprises a buffer. In another embodiment, the buffer comprises the following components: (i) 5% dimethyl sulfoxide (DMSO); (ii) 0.85M Betaine; (iii) 70 mM tetramethylammonium chloride (TMAC); (iv) 2.1 mM dATP; (v) 2.25 mM MgCl2; and (vi) 15 mM ammonium sulfate.

### EXAMPLES

### Example 1. Using Ethanol as Co-Solvent of Picoline-Borane in Methylation Detection Assays (TAPS)

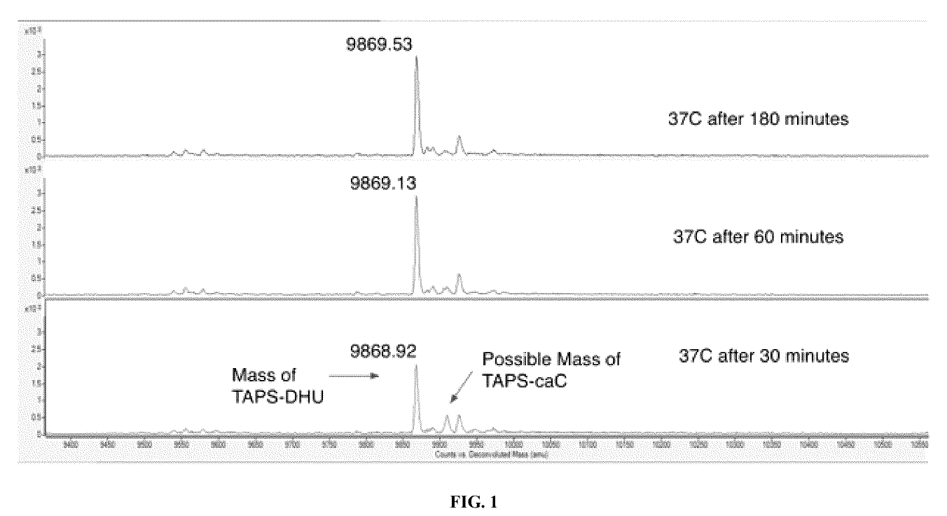

In this example, a synthetic oligonucleotide with a caC nucleotide was subjected to reduction by DHU under novel conditions. 2-Picoline borane was dissolved in absolute ethanol or methanol (1 mg/5 uL, 1.87 mM). The synthetic oligonucleotide, TAPS-caC SEQ ID NO: 1 (2.7 nmol, 5′-Phos-CACGTCCAGATCAAT(caC)GACTATGAGCAGTACA), was dissolved in 35 μL of sodium acetate (3M, pH 4.3) and mixed with 25 μL of picoline borane solution to a final concentration of 790 mM. The resulting cloudy solution was placed on a thermomixer shaking at 35 C for 3 hours. The solution was diluted with DI-water (300 uL) and was purified by HPLC (C18, eluents: CH3CN/0.1M TEAA; gradient: 2-15% CH3CN/0.1M TEAA in 35 minutes) to give 1.33 nmol (49%) of TAPS-DHU as identified by mass spectrometer (m/z 9869.43, calculated 9869.0). DHU product formation was monitored by taking an aliquot of reaction solution (2 uL) in period of 30, 60, 180 minutes and analyzed by LCMS. The results shown in FIG. 1 demonstrate that DHU conversion completed in an hour.

### Example 2. Using Methanol and Acetic Acid as Co-Solvents of Picoline-Borane in Methylation Detection Assays (TAPS)

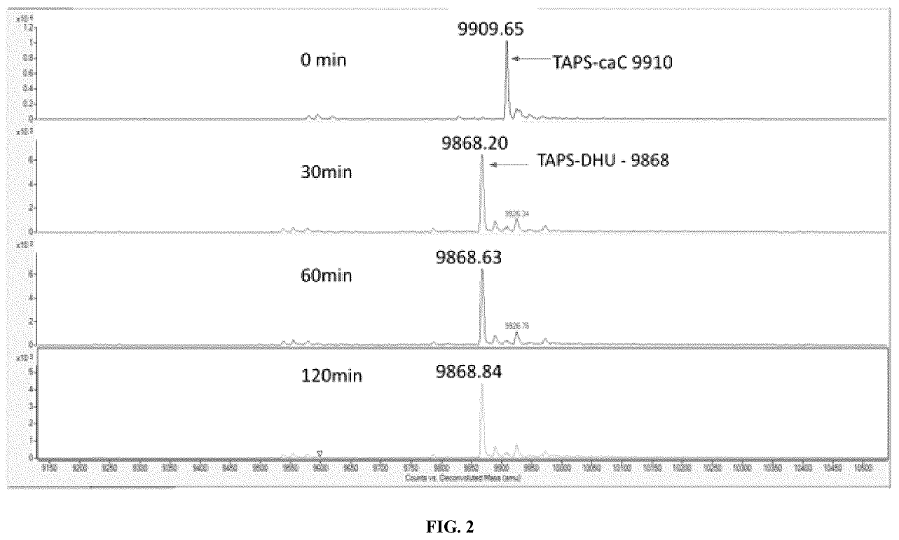

In this example, the oligonucleotide of Example 1 was used under the conditions described in Example 1 (SEQ ID NO: 1), except picoline-borane was present in methanol/acetic acid solution. The reaction contained 1.39 nmol TAPS caC oligonucleotide, 250 mM picborane in 10:1 v:v acetic acid and 200 mM MES pH 6. We were able to reduce the effective borane concentration to as low as 25 mM, and reaction time reduced further to 1 hr. The products of the reaction were detected as described in Example 1. Results are shown in FIG. 2.

### Example 3. Using Malononitrile in a Sodium Acetate Buffer in Methylation Detection Assays

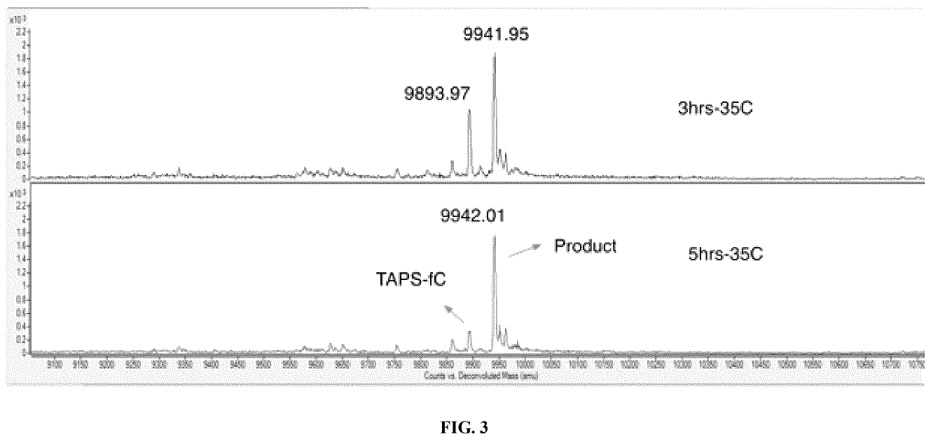

In this example, we demonstrate an improved process of malononitrile forming the adduct on the 5fC oligo at 35 C in 5 hrs using 1M sodium acetate as buffer. The reaction was performed as follows: 1 nmol (4 uL) of 5fC containing oligo (TAPS-fC: SEQ ID NO: 1 5′-Phos-CACGTCCAGATCAAT(fC)GACTATGAGCAGTACA)), reacts with malononitrile solution (100 mM, 50 uL) in sodium acetate (1M, pH 8.4). LCMS analysis of the sample at different incubation times show more than 90% of TAPS-fC was consumed after 5 hrs at 35 C. Results are shown in FIG. 3. Mass of TAPS-fC is 9894 and mass of the reacted product is 9942.

### Example 4. Using Malononitrile in an Ethanol-TRIS Buffer in Methylation Detection Assays

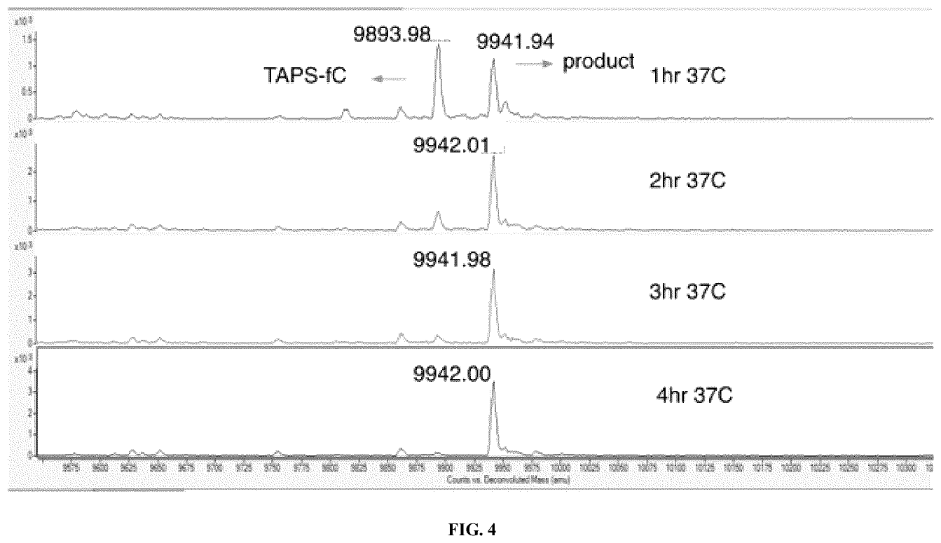

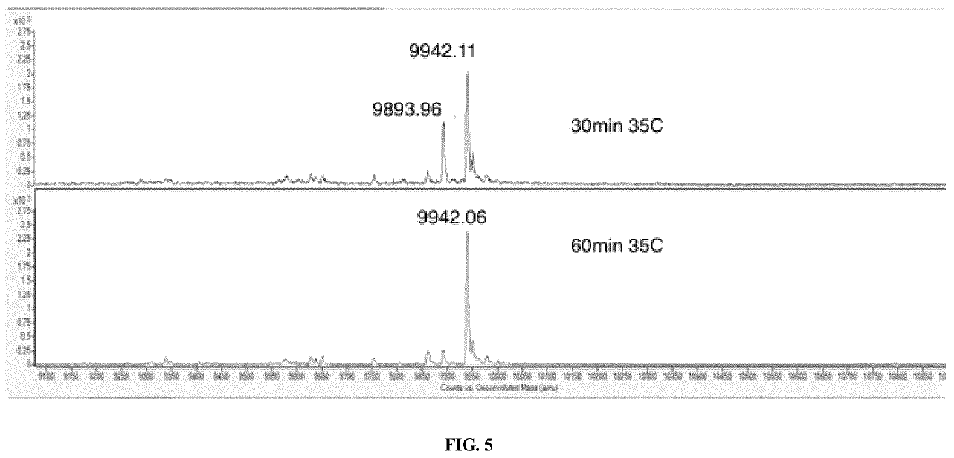

In this example, we demonstrate an improved process of malononitrile forming the adduct on the 5fC oligo at 35 C in 4 hrs using ethanol/TRIS as buffer. The reaction was performed as follows: 1 nmol (4 uL) of TAPS-fC oligo SEQ ID NO: 1 reacts with malononitrile solution (200 mM, 25 uL) in ethanol and TRIS (pH 8, 20 mM, 26 uL). LCMS analysis of the sample at different incubation time show most of 5fC was converted to product after 4 hours at 37 C. Results are shown in FIG. 4. Mass of TAPS-fC is 9894 and mass of the reacted product is 9942.

### Example 5. Using Malononitrile in an Ethanol-Triethylamine Buffer in Methylation Detection Assays

In this example, we demonstrate an improved process of malononitrile forming the adduct on the 5fC oligo at 35 C in an hour using ethanol/triethylamine as buffer. The reaction was performed as follows: 1 nmol (4 uL) of TAPS-5fC oligo (SEQ ID NO: 1) reacts with malononitrile solution (100 mM, 46 uL) in ethanol and 1 uL of triethylamine. LCMS analysis of the sample at different incubation time. More than 90% of the oligo reacted after 1 hr at 35 C. Results are shown in FIG. 5. Mass of TAPS-fC is 9894 and mass of the reacted product is 9942.

### Example 6. A Single-Tube Methylation Detection Assay with TET and Malononitrile

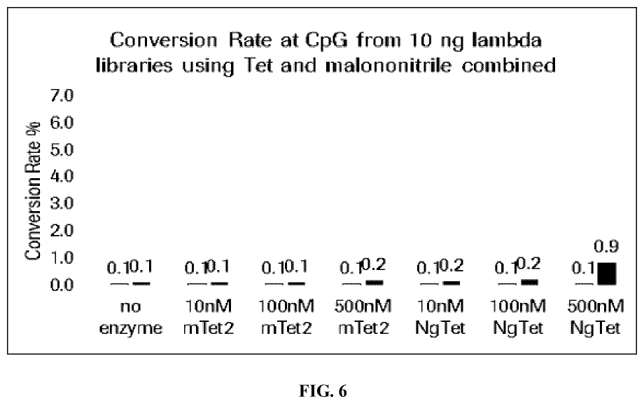

In this example, we demonstrate that TET is active in malononitrile enabling a single-tube methylation detection reaction. Lambda DNA (unmethylated and methylated) was sheared and ligated to adapters to make sequencing libraries. 10 ng of unmethylated and methylated libraries was used as input DNA. 10 nM, 100 nM or 500 nM of mTET2 (NEB) or NgTET was incubated with the DNA in a final concentration of 150 mM malononitrile. The reaction was incubated at 37 C for 20 hrs. A quarter of the sample was added directly into PCR for 14 cycles using Kapa HiFi polymerase. The libraries were sequenced and the rate of conversion at CpG sites is shown in FIG. 6. The conversion rate of 0.9% compared to no TET control of 0.1%, suggests that TET is able to function in the presence of malononitrile and oxidation and adduct formation can take place in a single tube.

### Example 7. Oxidation of 5hmC with Laccase Enzyme

In this example, a synthetic oligonucleotide with a 5hmC nucleotide was subjected to oxidation with laccase. The 5hmC synthetic oligonucleotide (SEQ ID NO: 2) had the sequence 5′-ATT ATT TAT TTA TThmC GTA TTA TTT ATT ATT-3′. The 150 μl reaction mixture comprised 50 nmol oligonucleotide, 1.6 mg (10 mol) 2,2,6,6-tetramethylpiperidine-1-oxyl (TEMPO), 2 mg laccase from Trametes versicolor (Sigma, Cat. No. 38429), 50 mM phosphate buffer pH 5.2. The reaction was allowed to proceed for 5 hrs at r.t. The first control reaction contained all the above reagents (including TEMPO) except laccase. The second control reaction contained all the above reagents (including laccase and TEMPO) except laccase and TEMPO were added at 1:1000 dilution. The third control reaction contained all the above reagents except the oligonucleotide SEQ. ID. NO: 2 contained 5mC instead of 5hmC: 5′-ATT ATT TAT TTA TTmC GTA TTA TTT ATT ATT-3′.

The reaction mixture was analyzed by liquid chromatography-mass-spectrometry (LCMS). The molecular weights are as follows:
5hmC—9176 Da
5mC—9160 Da
5fC—9174 Da

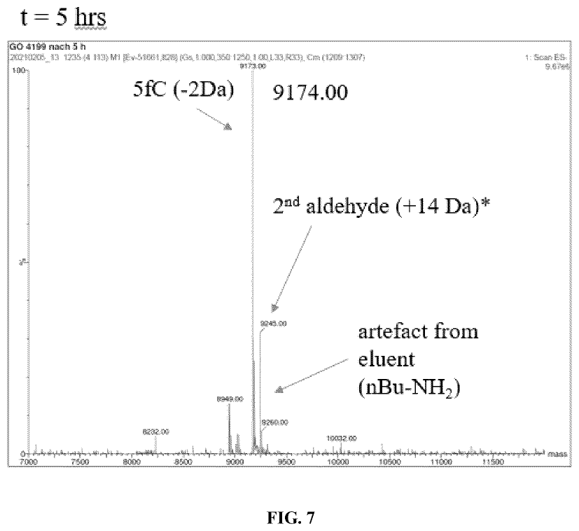

FIG. 7 shows the 5fC peak at 9174 Da resulting from the reaction. An additional peak is observed at 9229 Da corresponding to an artefact resulting from imine formation of 5-formyl-dC with n-butylamine from LC-MS eluent (fC+55 Da=9229 Da). To some extent, an additional oxidation occurs at the hydroxyl group of the ribose sugar (+14 Da).

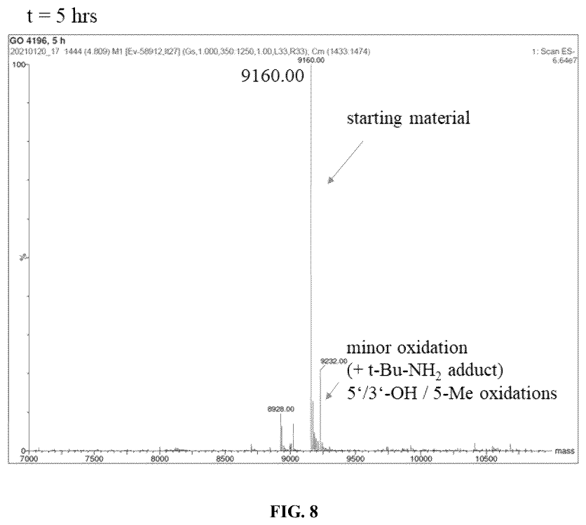

FIG. 8 shows no reaction with 5mC (5mC peak unchanged at 9160 Da). To some extent, an oxidation occurs at the hydroxyl group of the ribose sugar (+14 Da). Control reactions with no enzyme and diluted enzyme showed no change in the 5hmC starting material (data not shown).

### Example 8 (Prophetic). Oxidizing 5mC into 5hmC Prior to Reacting 5hmC with Laccase

To convert 5mC to 5hmC, the reaction mixture contains 3 ug TET protein and 2 g of oligonucleotide substrates in 50 mM HEPES, pH 8, 50 mM NaCl, 2 mM Ascorbic Acid, 1 mM 2-oxoglutarate, 10 μM ferrous ammonium sulfate (Fe2+), and 1 mM DTT and is incubated for 3 hours at 37° C. as described by Tahiliani et al, (2009) Conversion of 5-Methylcytosine to 5-Hydroxymethylcytosine in Mammalian DNA by MLL Partner TET1, Science 324 (5929):930-935. The resulting nucleic acid is purified e.g., by SPRI. An aliquot of the reaction mixture is incubated with laccase as described in Example 7.

While the invention has been described in detail with reference to specific examples, it will be apparent to one skilled in the art that various modifications can be made within the scope of this invention. Thus the scope of the invention should not be limited by the examples described herein, but by the claims presented below.

### Example 9: Use of Amine Buffer Catalysts to Modulate TET Activity to 5hmC/5fC

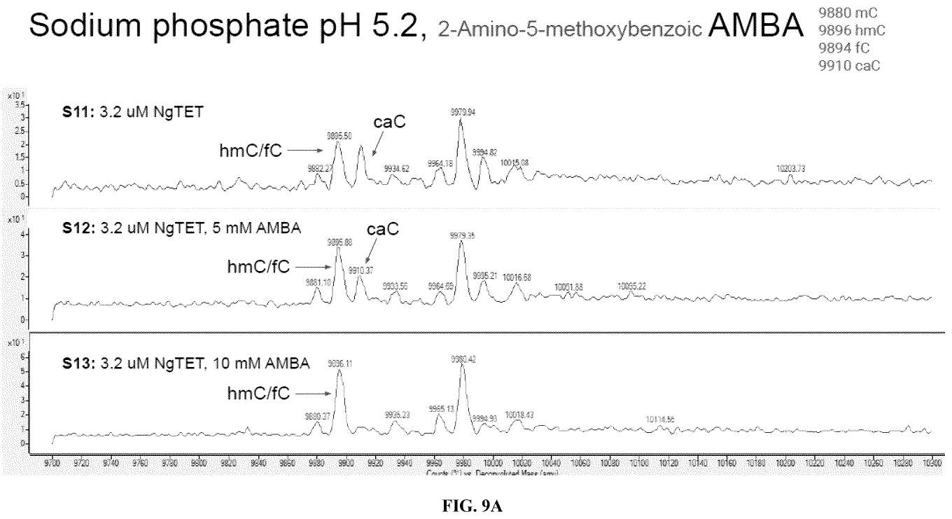

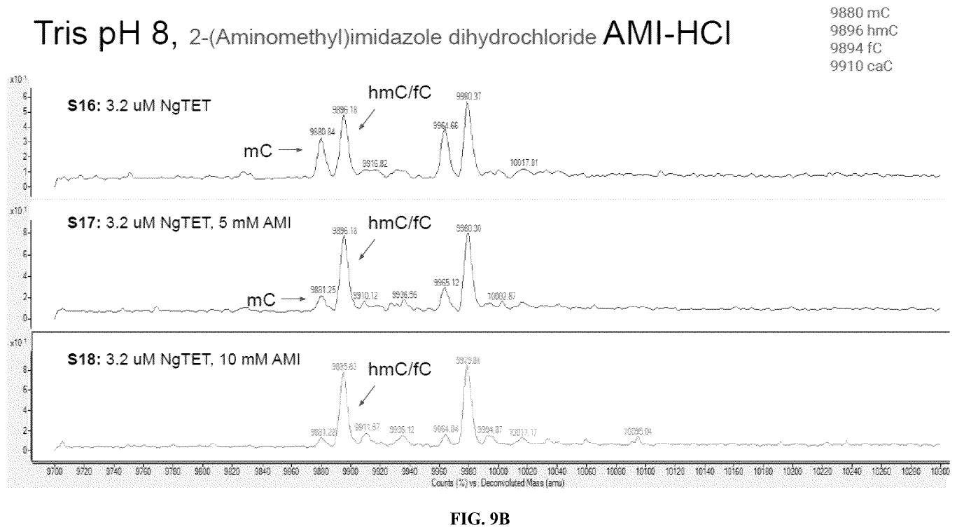

Studies were conducted to determine if amine buffer catalysts could modulate TET activity to oxidize 5mC to 5hmC/5fC. To study this, 1 μg of duplex DNA containing 5mC were oxidized with 3. μM NgTET at 37° C., for 1 hour, in the presence or absence of catalysts in certain buffer conditions. In one experiment, the catalyst, 2-Amino-5-methoxybenzoic acid (AMBA) was added to modulate TET activity in a buffer containing sodium phosphate, and having a pH of 5.2 (results depicted in FIG. 9A). The top graph of FIG. 9A shows TET without any AMBA, the middle graph of FIG. 9A shows TET with 5 mM AMBA, and the bottom graph of FIG. 9A shows TET with 10 mM AMBA. FIG. 9A shows that AMBA promotes TET-mediated oxidation of 5mC to 5hmC/5fC efficiently, in a dose-dependent manner, without undue accumulation of unwanted 5caC product. In another experiment, the catalyst, 2-(Aminomethyl)imidazole dihydrochloride (AMI) was added to modulate TET activity in a buffer containing Tris, and having a pH of 8 (results depicted in FIG. 9B). The top graph of FIG. 9B shows TET without any AMI, the middle graph of FIG. 9B shows TET with 5 mM AMI, and the bottom graph of FIG. 9B shows TET with 10 mM AMI. FIG. 9B shows that AMI promotes TET-mediated oxidation of 5mC to 5hmC/5fC efficiently, in a dose-dependent manner, without undue accumulation of unwanted 5caC product. Thus, these studies demonstrate that amine catalysts, AMBA and AMI, in a buffer, in the presence of TET, favor the oxidation of 5mC to 5hmC/5fC.

Taken together, these studies suggest that amine catalysts (such as 2-Amino-5-methoxybenzoic acid (AMBA) and 2-(Aminomethyl)imidazole dihydrochloride (AMI) maybe useful to promote TET-mediated oxidation of 5mC to favor generation of 5hmC and 5fC species, while discouraging full oxidation to the 5caC species. Thus, amine catalysts (such as 2-Amino-5-methoxybenzoic (AMBA) and 2-(Aminomethyl)imidazole dihydrochloride (AMI) maybe ultimately be useful and employed in workflows to detect 5mC and 5hmC species by ultimately promoting Thymine production.

### Example 10: Detection and Accumulation of 5fC Species by Oxidation of 5hmC with Laccase Enzyme as Early as within 3 Hours

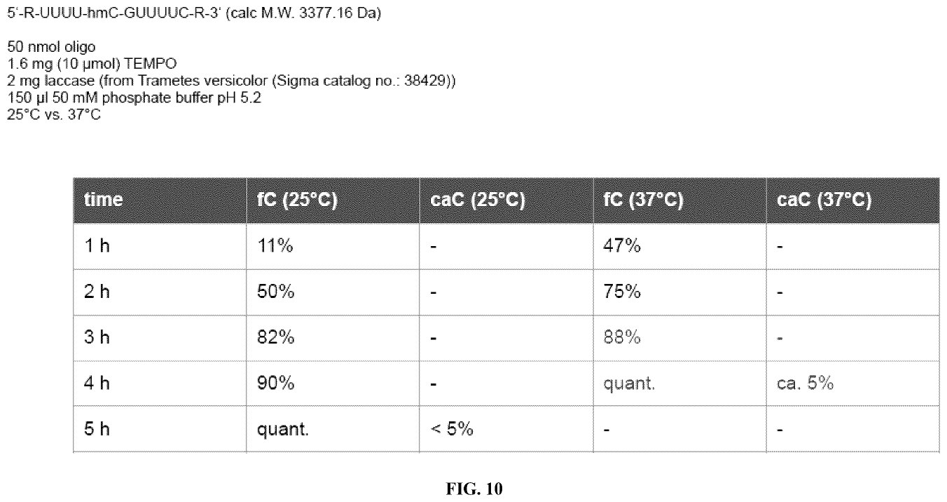

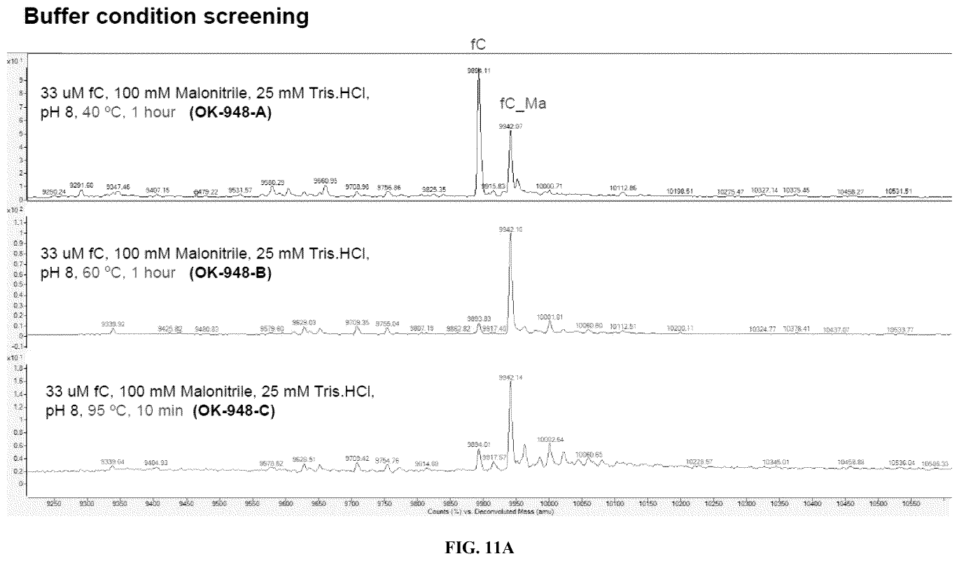

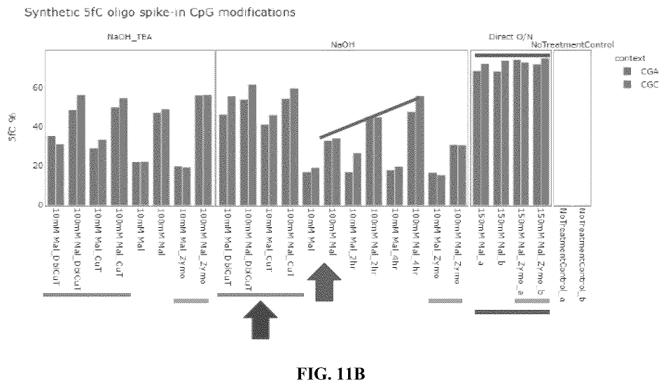

Studies show that laccase enzyme converts 5hmC to 5fC after 22 hours. Studies were conducted in order to determine if 5fC could be detected earlier/faster than 22 hours. To that end, the ability of laccase (2 mg laccase (from Trametes versicolor (Sigma Catalog No. 38429)) to oxidize 5hmC from 50 nM of a 5hmC-containing oligonucleotide substrate was tested, in the presence of 1.6 mg (1 μM), 2,2,6,6-tetramethylpiperidine-1-oxyl (TEMPO), and 150 μl of 50 mM phosphate buffer (pH 5.2), at two different temperatures (25° C. and 37° C.). The sequence of the 5hmC-containing oligonucleotide employed is shown in FIG. 10. FIG. 10 shows that significant amounts of 5fC generated/accumulated by the laccase oxidation of 5hmC can be detected as early as 3 hours, at 25° C. and 37° C.

These studies demonstrate that laccase is efficient at converting 5hmC to 5fC, within as early as 3 hours, at either 25° C. and 37° C., without the unwanted accumulation/conversion of unwanted 5caC (as shown in FIG. 10). Thus, these studies show that laccase can be employed as an important enzyme for detecting methylated species (e.g., 5hmC) by promoting the conversion to 5fC, which can then be subsequently converted to Thymine.

### Example 11: Optimization of Malononitrile Activity in Converting 5fC to 5fC-M Adduct

Studies were conducted here, to optimize malononitrile activity in converting 5fC to 5fC-M adduct.

In particular, studies were conducted here to determine if elevated temperatures could accelerate the reaction of malononitrile-mediated conversion of 5fC to 5fC-M adduct. Earlier studies in the art have demonstrated that malononitrile can be employed to convert 5fC to a 5fC-M adduct, however, these studies were conducted at 37° C., in 10 mM Tris, for 20 hours (see, U.S. Pat. No. 10,519,184 and U.S. Patent Publication No. US 2020/0165661). To assess the effect of elevated temperatures, 33 μM of 5fC was added to 100 mM malononitrile, in 25 mM Tris HCl, at different temperatures and incubation times (40° C. for 1 hour, 60° C. for 1 hour, and 95° C. for 10 minutes). Results are shown in FIG. 11A. FIG. 11A shows LC-MS data of the effect of malononitrile on conversion of 5fC to 5fC-M adduct under various buffer conditions. The top graph of FIG. 11A shows buffer conditions of 40° C. for 1 hour, the middle graph of FIG. 11A shows buffer conditions of 60° C. for 1 hour, and the bottom graph of FIG. 11A shows buffer conditions of 95° C. for 10 minutes. FIG. 11A shows dramatic accumulation of 5fC-M adduct at elevated temperatures (60° C.) after 1 hour. FIG. 11A also shows dramatic accumulation of 5fC-M adduct at elevated temperatures (95° C.) after only 10 minutes (but with the existence of degradation products). Thus, FIG. 11A shows that malononitrile activity in converting 5fC to 5fC-M adduct is optimized at elevated temperatures (60° C.), which represents a significant improvement over the art.

In another study, the effect of NaOH, which pre-denatures double-stranded nucleic acids, on malononitrile activity of converting 5fC to 5fC-M adduct conversion, was assessed. To that end, two different synthetic 5fC oligonucleotides (CGA and CGC) were employed as substrates, in the presence of malononitrile and NaOH in copper(II) perchlorate and 2,2,6,6-tetramethylpiperidine-1-oxyl (TEMPO) (Cu(II)/TEMPO), in 30 minutes. Malononitrile activity in converting 5fC to 5fC-M adduct was assessed, and results are depicted in FIG. 11B. FIG. 11B shows significant malononitrile activity in the presence of NaOH in as soon as 30 minutes. These studies demonstrate that malononitrile activity is enhanced with NaOH pre-denaturation.

Additionally, it likely that malononitrile activity can be optimized also with a shortened incubation time (1.5 hours) on single-stranded DNA using 25 mM Tris buffer, at pH of 8, at 60° C. It is believed that elevated/increasing amounts of Tris (e.g., 25 mM Tris), and at elevated temperatures (e.g., 50 C-60 C), would result in an acceleration of the reaction, and improved malononitrile activity efficiencies (data not shown). Furthermore, it is also likely that the addition of copper(II) perchlorate and 2,2,6,6-tetramethylpiperidine-1-oxyl (TEMPO) (Cu(II)/TEMPO) components can enhance reaction efficiency (data not shown).

Taken together, these studies demonstrate that malononitrile activity can be optimized by: (i) increasing incubation temperature (to 60° C.), (ii) shortening incubation times (to 1.5 hours) on single-stranded DNA by employing buffers high in Tris (e.g., 25 mM Tris), with a pH of 8, (iii) employing a pre-denaturation step using NaOH (which can shorten the incubation on double-stranded DNA), and (iv) the addition of copper(II) perchlorate and 2,2,6,6-tetramethylpiperidine-1-oxyl (Cu(II)/TEMPO) components (which can enhance reaction efficiencies). Thus, the ability to detect methylated species in oligonucleotide samples can be improved/enhanced by optimizing malononitrile activity in a number of ways.

### Example 12: Optimization of Copper(II) Perchlorate and 2,2,6,6-tetramethylpiperidine-1-oxyl (Cu(II)/TEEMPO)

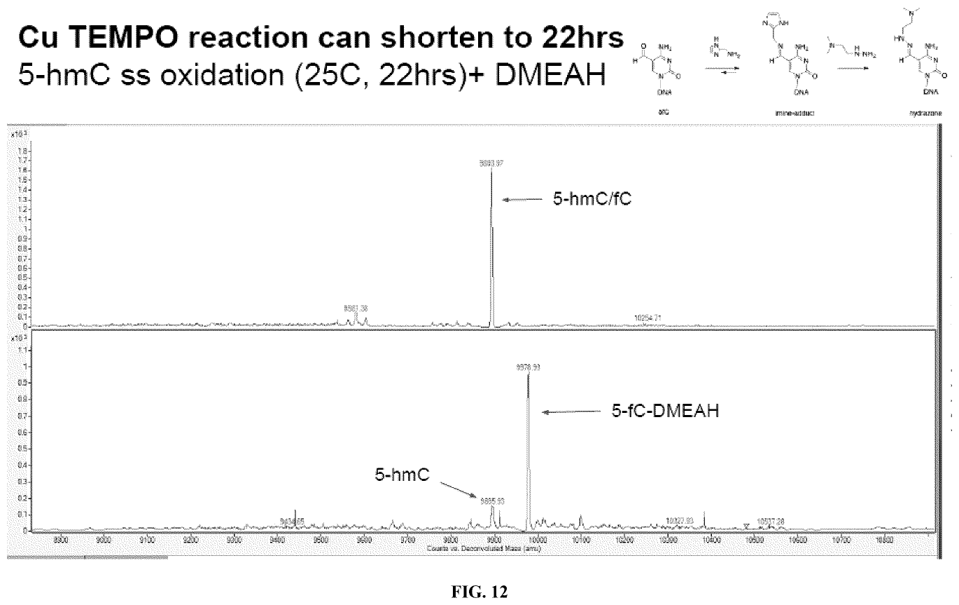

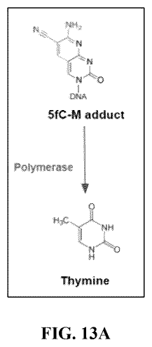

Studies were conducted to optimize copper(II) perchlorate and 2,2,6,6-tetramethylpiperidine-1-oxyl (Cu(II)/TEMPO) reaction conditions to assess its effects on CuTEMPO-mediated oxidation of 5hmC to 5fC. Earlier studies in the art demonstrated that Cu(II)/TEMPO reactions occur at room temperature and for as long as 48 hours (see, e.g., Matsushita, et al., “DNA-friendly Cu(II)/TEMPO-catalyzed 5-hydroxymethylcytosine-specific oxidation,” Chem. Commun. 53:5756-5759 (2017)). These studies were conducted to assess if the Cu(II)/TEMPO reaction could be shortened to 22 hours. To assess this, the following reagents were combined: 49 μl of H₂O, 10 μl of Cu(ClO₄)₂ (100 mM), 15 d of BiPyr (100 mM), 10 μl of TEMPO (100 mM), 10 μl of NaOH (50 mM), and 6 μl of hMC (16 μM), for 22 hours, at 25° C., mixed at 500 rpm, in the presence or absence of dimethylaminoethylhydrazine (DMAEH) (which only reacts on 5fC). The results are depicted in FIG. 12. The top graph of FIG. 12 shows Cu(II)/TEMPO oxidation of 5mC to 5hmC/5fC, and the bottom graph of FIG. 12 shows the derivatization of the product from the top graph using DMAEH.

Thus, these data show that copper(II) perchlorate and 2,2,6,6-tetramethylpiperidine-1-oxyl (Cu(II)/TEMPO) reactions can be shortened to 22 hours, which represents an improvement over the art.

### Example 13: Buffer Conditions to Optimize Polymerase Activity for Conversion of 5fC-M Adduct to Thymine (T)

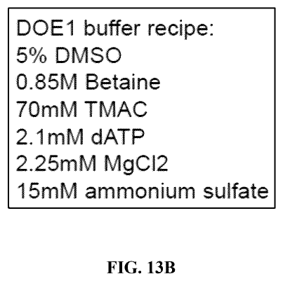

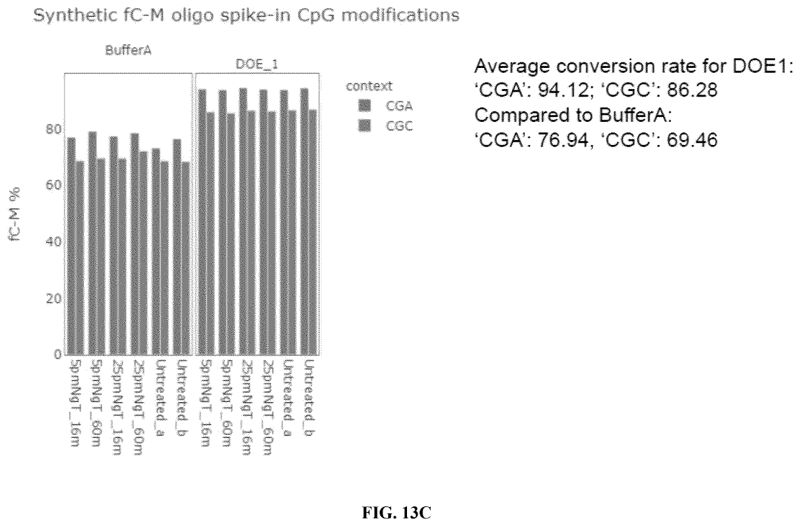

Polymerase enzymes mediate the step of converting 5fC-M adduct to Thymine (T), as depicted in FIG. 13A. Studies were conducted to assess the effect of buffer on the conversion of 5fC-M adduct to T. To that end, standard buffer (“BufferA”) was compared with an optimized buffer (“DOE_1”). The components of optimized Buffer DOE_1 are shown in FIG. 13B. Oligonucleotides containing a purified 5fC-M adduct in a “CGA” or “CGC” context are amplified with polymerase in standard buffer (“BufferA”) or optimized buffer (“DOE_1”). FIG. 13C shows the data showing conversion of 5fC-M adduct to T using standard buffer (“BufferA”) and an optimized buffer (“DOE_1”). In particular, FIG. 13C shows that the conversion of 5fC-M adduct to T is increased/enhanced with the optimized buffer (“DOE_1”) compared to standard buffer. Indeed, as FIG. 13C shows, for “CGA,” the conversion rate with the optimized buffer (94.12) is significantly greater than the conversion rate with the standard buffer (76.94), and for “CGC,” the conversion rate with the optimized buffer (86.28) is significantly greater than the conversion rate with the standard buffer (69.46). Thus, these studies demonstrate that the polymerase activity is enhanced in the presence of optimized buffer as compared to standard buffer.

Taken together, these studies show that the polymerase activity of converting 5fC-M adduct to Thymine (T) can be enhanced with optimized buffer. This means that the methods for detecting methylated species in oligonucleotide samples can be improved using improved buffer for improved polymerase activity.

1-124. (canceled)

125. A method for detecting 5-hydroxymethylcytosine (5hmC) in a target nucleic acid from a sample, wherein the method comprises the following steps:
(a) contacting the target nucleic acid with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC);
(b) contacting the nucleic acid comprising one or more 5fC of step (a) with malononitrile, wherein the malononitrile converts 5fC to 5fC-M adduct, thereby producing a nucleic acid comprising one or more 5fC-M adduct;
(c) contacting the nucleic acid comprising one or more 5fC-M adduct of step (b) with a polymerase, wherein the polymerase converts 5fC-M adduct to Thymine (T), thereby producing a nucleic acid comprising one or more T; and
(d) sequencing the nucleic acid comprising one or more T of step (c), wherein if a T is detected at a position in the nucleic acid comprising one or more T of step (c) where a 5hmC was originally present in the target nucleic acid, then 5hmC has been detected in the target nucleic acid.

126. The method of claim 125, wherein the target nucleic acid is contacted with laccase in step (a).

127. The method of claim 126, wherein step (a) occurs in less than 22 hours.

128. The method of claim 127, wherein step (a) occurs in less than 5 hours.

129. The method of claim 128, wherein step (a) occurs in less than 4 hours.

130. The method of claim 129, wherein step (a) occurs in less than 3 hours.

131. The method of claim 126, wherein step (a) occurs in 3 hours.

132. The method of claim 126, wherein step (a) occurs at around 25° C.

133. The method of claim 126, wherein step (a) occurs at 25° C.

134. The method of claim 126, wherein step (a) occurs at around 37° C.

135. The method of claim 126, wherein step (a) occurs at 37° C.

136. The method of claim 125, wherein the target nucleic acid is contacted with Cu(II)/TEMPO in step (a).

137. The method of claim 136, wherein step (a) occurs in less than 24 hours.

138. The method of claim 137, wherein step (a) occurs in 22 hours.

139. The method of claim 125, wherein step (b) occurs at around 60° C.

140. The method of claim 125, wherein step (b) occurs at 60° C.

141. The method of claim 140, wherein step (b) occurs in a reaction mixture, wherein the reaction mixture comprises a buffer.

142. The method of claim 141, wherein the buffer comprises 25 mM Tris.

143. The method of claim 141, wherein the buffer is at a pH of around 8.

144. The method of claim 141, wherein the nucleic acid comprising one or more 5fC is contacted with malononitrile for 1.5 hours.

145. The method of claim 125, further comprising an additional step between step (a) and step (b), wherein the additional step between step (a) and step (b) comprises contacting the nucleic acid comprising one or more 5fC with NaOH.

146. The method of claim 145, wherein the nucleic acid comprising one or more 5fC is contacted with malononitrile in step (b) for less than 1 hour.

147. The method of claim 145, wherein the nucleic acid comprising one or more 5fC is contacted with malononitrile in step (b) for about 1 hour.

148. The method of claim 145, wherein the nucleic acid comprising one or more 5fC is contacted with malononitrile in step (b) for less than 30 minutes.

149. The method of claim 145, wherein the nucleic acid comprising one or more 5fC is contacted with malononitrile in step (b) for about 30 minutes.

150. The method of claim 125, wherein step (c) occurs in a reaction mixture, wherein the reaction mixture comprises a buffer.

151. The method of claim 150, wherein the buffer comprises the following components:
(i) 5% dimethyl sulfoxide (DMSO);
(ii) 0.85M Betaine;
(iii) 70 mM tetramethylammonium chloride (TMAC);
(iv) 2.1 mM dATP;
(v) 2.25 mM MgCl2; and
(vi) 15 mM ammonium sulfate.

152. A method for detecting 5-methylcytosine (5mC) in a target nucleic acid from a sample, wherein the method comprises the following steps:
(a) contacting the target nucleic acid with ten-eleven-translocation (TET), wherein the TET converts 5mC to 5-hydroxymethylcytosine (5hmC), thereby producing a nucleic acid comprising one or more 5hmC;
(b) contacting the nucleic acid comprising one or more 5hmC of step (a) with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC);
(c) contacting the nucleic acid comprising one or more 5fC of step (b) with malononitrile, wherein the malononitrile converts 5fC to 5fC-malononitrile adduct (55fC-M adduct), thereby producing a nucleic acid comprising one or more 5fC-M adduct;
(d) contacting the nucleic acid comprising one or more 5fC-M adduct of step (c) with a polymerase, wherein the polymerase converts 5fC-M adduct to Thymine (T), thereby producing a nucleic acid comprising one or more T; and
(e) sequencing nucleic acid comprising one or more T of step (d), wherein if a T is detected at a position in the nucleic acid comprising one or more T of step (d) where a 5hmC was originally present in the target nucleic acid, then 5hmC has been detected in the target nucleic acid.

153. The method of claim 152, wherein step (a) occurs in a reaction mixture, wherein the reaction mixture comprises a buffer comprising an amine catalyst.

154. The method of claim 153, wherein the amine catalyst is 2-amino-5-methoxybenzoic acid.

155. The method of claim 154, wherein the buffer comprises sodium phosphate and has a pH of around 5.2.

156. The method of claim 153, wherein the amine catalyst is 2-(aminomethyl)imidazole dihydrochloride.

157. The method of claim 156, wherein the buffer comprises Tris and has a pH of around 8.

158. The method of claim 152, wherein the target nucleic acid is contacted with laccase in step (b).

159. The method of claim 158, wherein step (b) occurs in less than 22 hours.

160. The method of claim 159, wherein step (b) occurs in less than 5 hours.

161. The method of claim 160, wherein step (b) occurs in less than 4 hours.

162. The method of claim 161, wherein step (b) occurs in less than 3 hours.

163. The method of claim 158, wherein step (b) occurs in 3 hours.

164. The method of claim 158, wherein step (b) occurs at around 25° C.

165. The method of claim 158, wherein step (b) occurs at 25° C.

166. The method of claim 158, wherein step (b) occurs at around 37° C.

167. The method of claim 158, wherein step (b) occurs at 37° C.

168. The method of claim 158, wherein step (a) and step (b) are combined in a single step.

169. The method of claim 168, wherein the combination of step (a) and step (b) occurs in a reaction mixture, wherein the reaction mixture comprises a buffer, and wherein the buffer comprises both TET and laccase.

170. The method of claim 169, wherein the target nucleic acid is contacted with TET, wherein the TET converts 5mC to 5hmC, thereby producing a nucleic acid comprising one or more 5hmC; and wherein the laccase converts 5hmC to 5fC, thereby producing a nucleic acid comprising one or more 5fC.

171. The method of claim 152, wherein the target nucleic acid is contacted with Cu(II)/TEMPO in step (b).

172. The method of claim 171, wherein step (b) occurs in less than 24 hours.

173. The method of claim 172, wherein step (b) occurs in 22 hours.

174. The method of claim 152, wherein step (c) occurs at around 60° C.

175. The method of claim 152, wherein step (c) occurs at 60° C.

176. The method of claim 175, wherein step (c) occurs in a reaction mixture, wherein the reaction mixture comprises a buffer.

177. The method of claim 176, wherein the buffer comprises 25 mM Tris.

178. The method of claim 176, wherein the buffer is at a pH of around 8.

179. The method of claim 176, wherein the nucleic acid comprising one or more 5fC is contacted with malononitrile for 1.5 hours.

180. The method of claim 152, further comprising an additional step between step (b) and step (c), wherein the additional step between step (b) and step (c) comprises contacting the nucleic acid comprising one or more 5fC with NaOH.

181. The method of claim 180, wherein the nucleic acid comprising one or more 5fC is contacted with malononitrile in step (c) for less than 1 hour.

182. The method of claim 180, wherein the nucleic acid comprising one or more 5fC is contacted with malononitrile in step (c) for about 1 hour.

183. The method of claim 180, wherein the nucleic acid comprising one or more 5fC is contacted with malononitrile in step (c) for less than 30 minutes.

184. The method of claim 180, wherein the nucleic acid comprising one or more 5fC is contacted with malononitrile in step (c) for about 30 minutes.

185. The method of claim 152, wherein step (d) occurs in a reaction mixture, wherein the reaction mixture comprises a buffer.

186. The method of claim 185, wherein the buffer comprises the following components:
(i) 5% dimethyl sulfoxide (DMSO);
(ii) 0.85M Betaine;
(iii) 70 mM tetramethylammonium chloride (TMAC);
(iv) 2.1 mM dATP;
(v) 2.25 mM MgCl2; and
(vi) 15 mM ammonium sulfate.

187. A method for converting 5-hydroxymethylcytosine (5hmC), in a target nucleic acid from a sample, to Thymine (T), wherein the method comprises the following steps:
(a) contacting the target nucleic acid with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC);
(b) contacting the nucleic acid comprising one or more 5fC of step (a) with malononitrile, wherein the malononitrile converts 5fC to 5fC-M adduct, thereby producing a nucleic acid comprising one or more 5fC-M adduct; and
(c) contacting the nucleic acid comprising one or more 5fC-M adduct of step (b) with a polymerase, wherein the polymerase converts 5fC-M adduct to Thymine (T), thereby producing a nucleic acid comprising one or more T; and
wherein the 5hmC in a target nucleic acid has been converted to T.

188. The method of claim 187, wherein the target nucleic acid is contacted with laccase in step (a).

189. The method of claim 188, wherein step (a) occurs in less than 22 hours.

190. The method of claim 189, wherein step (a) occurs in less than 5 hours.

191. The method of claim 190, wherein step (a) occurs in less than 4 hours.

192. The method of claim 191, wherein step (a) occurs in less than 3 hours.

193. The method of claim 188, wherein step (a) occurs in 3 hours.

194. The method of claim 188, wherein step (a) occurs at around 25° C.

195. The method of claim 188, wherein step (a) occurs at 25° C.

196. The method of claim 188, wherein step (a) occurs at around 37° C.

197. The method of claim 188, wherein step (a) occurs at 37° C.

198. The method of claim 187, wherein the target nucleic acid is contacted with Cu(II)/TEMPO in step (a).

199. The method of claim 198, wherein step (a) occurs in less than 24 hours.

200. The method of claim 199, wherein step (a) occurs in 22 hours.

201. The method of claim 187, wherein step (b) occurs at around 60° C.

202. The method of claim 187, wherein step (b) occurs at 60° C.

203. The method of claim 202, wherein step (b) occurs in a reaction mixture, wherein the reaction mixture comprises a buffer.

204. The method of claim 203, wherein the buffer comprises 25 mM Tris.

205. The method of claim 203, wherein the buffer is at a pH of around 8.

206. The method of claim 203, wherein the nucleic acid comprising one or more 5fC is contacted with malononitrile for 1.5 hours.

207. The method of claim 187, further comprising an additional step between step (a) and step (b), wherein the additional step between step (a) and step (b) comprises contacting the nucleic acid comprising one or more 5fC with NaOH.

208. The method of claim 207, wherein the nucleic acid comprising one or more 5fC is contacted with malononitrile in step (b) for less than 1 hour.

209. The method of claim 207, wherein the nucleic acid comprising one or more 5fC is contacted with malononitrile in step (b) for about 1 hour.

210. The method of claim 207, wherein the nucleic acid comprising one or more 5fC is contacted with malononitrile in step (b) for less than 30 minutes.

211. The method of claim 207, wherein the nucleic acid comprising one or more 5fC is contacted with malononitrile in step (b) for about 30 minutes.

212. The method of claim 187, wherein step (c) occurs in a reaction mixture, wherein the reaction mixture comprises a buffer.

213. The method of claim 212, wherein the buffer comprises the following components:
(i) 5% dimethyl sulfoxide (DMSO);
(ii) 0.85M Betaine;
(iii) 70 mM tetramethylammonium chloride (TMAC);
(iv) 2.1 mM dATP;
(v) 2.25 mM MgCl2; and
(vi) 15 mM ammonium sulfate.

214. A method for converting 5-methylcytosine (5mC), in a target nucleic acid from a sample, to Thymine (T), wherein the method comprises the following steps:
(a) contacting the target nucleic acid with ten-eleven-translocation (TET), wherein the TET converts 5mC to 5-hydroxymethylcytosine (5hmC), thereby producing a nucleic acid comprising one or more 5hmC;
(b) contacting the nucleic acid comprising one or more 5hmC of step (a) with laccase or copper(II) perchlorate and 2,2,6,6-tetramethylpiperidine-1-oxyl (Cu(II)/TEMPO), wherein the laccase or Cu(II)/TEMPO converts 5hmC to 5-formylcytosine (5fC), thereby producing a nucleic acid comprising one or more 5-formylcytosine (5fC);
(c) contacting the nucleic acid comprising one or more 5fC of step (b) with malononitrile, wherein the malononitrile converts 5fC to 5fC-malononitrile adduct (5fC-M adduct), thereby producing a nucleic acid comprising one or more 5fC-M adduct;
(d) contacting the nucleic acid comprising one or more 5fC-M adduct of step (c) with a polymerase, wherein the polymerase converts 5fC-M adduct to Thymine (T), thereby producing a nucleic acid comprising one or more T; and
wherein the 5mC in a target nucleic acid has been converted to T.

215. The method of claim 214, wherein step (a) occurs in a reaction mixture, wherein the reaction mixture comprises a buffer comprising an amine catalyst.

216. The method of claim 215, wherein the amine catalyst is 2-amino-5-methoxybenzoic acid.

217. The method of claim 216, wherein the buffer comprises sodium phosphate and has a pH of around 5.2.

218. The method of claim 215, wherein the amine catalyst is 2-(aminomethyl)imidazole dihydrochloride.

219. The method of claim 218, wherein the buffer comprises Tris and has a pH of around 8.

220. The method of claim 214, wherein the target nucleic acid is contacted with laccase in step (b).

221. The method of claim 220, wherein step (b) occurs in less than 22 hours.

222. The method of claim 221, wherein step (b) occurs in less than 5 hours.

223. The method of claim 222, wherein step (b) occurs in less than 4 hours.

224. The method of claim 223, wherein step (b) occurs in less than 3 hours.

225. The method of claim 220, wherein step (b) occurs in 3 hours.

226. The method of claim 220, wherein step (b) occurs at around 25° C.

227. The method of claim 220, wherein step (b) occurs at 25° C.

228. The method of claim 220, wherein step (b) occurs at around 37° C.

229. The method of claim 220, wherein step (b) occurs at 37° C.

230. The method of claim 220, wherein step (a) and step (b) are combined in a single step.

231. The method of claim 230, wherein the combination of step (a) and step (b) occurs in a reaction mixture, wherein the reaction mixture comprises a buffer, and wherein the buffer comprises both TET and laccase.

232. The method of claim 231, wherein the target nucleic acid is contacted with TET, wherein the TET converts 5mC to 5hmC, thereby producing a nucleic acid comprising one or more 5hmC; and wherein the laccase converts 5hmC to 5fC, thereby producing a nucleic acid comprising one or more 5fC.

233. The method of claim 214, wherein the target nucleic acid is contacted with Cu(II)/TEMPO in step (b).

234. The method of claim 233, wherein step (b) occurs in less than 24 hours.

235. The method of claim 234, wherein step (b) occurs in 22 hours.

236. The method of claim 214, wherein step (c) occurs at around 60° C.

237. The method of claim 214, wherein step (c) occurs at 60° C.

238. The method of claim 237, wherein step (c) occurs in a reaction mixture, wherein the reaction mixture comprises a buffer.

239. The method of claim 238, wherein the buffer comprises 25 mM Tris.

240. The method of claim 238, wherein the buffer is at a pH of around 8.

241. The method of claim 238, wherein the nucleic acid comprising one or more 5fC is contacted with malononitrile for 1.5 hours.

242. The method of claim 214, further comprising an additional step between step (b) and step (c), wherein the additional step between step (b) and step (c) comprises contacting the nucleic acid comprising one or more 5fC with NaOH.

243. The method of claim 242, wherein the nucleic acid comprising one or more 5fC is contacted with malononitrile in step (c) for less than 1 hour.

244. The method of claim 242, wherein the nucleic acid comprising one or more 5fC is contacted with malononitrile in step (c) for about 1 hour.

245. The method of claim 242, wherein the nucleic acid comprising one or more 5fC is contacted with malononitrile in step (c) for less than 30 minutes.

246. The method of claim 242, wherein the nucleic acid comprising one or more 5fC is contacted with malononitrile in step (c) for about 30 minutes.

247. The method of claim 214, wherein step (d) occurs in a reaction mixture, wherein the reaction mixture comprises a buffer.

248. The method of claim 247, wherein the buffer comprises the following components:
(i) 5% dimethyl sulfoxide (DMSO);
(ii) 0.85M Betaine;
(iii) 70 mM tetramethylammonium chloride (TMAC);
(iv) 2.1 mM dATP;
(v) 2.25 mM MgCl2; and
(vi) 15 mM ammonium sulfate.
